(19) United States
(12) Patent Application Publication
(10) Pub. No.: US 2012/0077836 A1
(43) Pub. Date: Mar. 29, 2012

(54) METHODS OF ADMINISTERING (4AR,10AR)-1-N-PROPYL-1,2,3,4,4A,5,10,10A-OCTAHYDROBENZO [G] QUINOLINE-6,7-DIOL AND RELATED COMPOUNDS ACROSS THE ORAL MUCOSA, THE NASAL MUCOSA OR THE SKIN AND PHARMACEUTICAL COMPOSITIONS THEREOF

(75) Inventors: Håkan Wikström, Hamburgsund (SE); Morten Jorgensen, Bagsvaerd (DK); Niels Mork, Virum (DK); Jennifer Larsen, Roskilde (DK); Benny Bang-Andersen, Copenhagen S (DK); Thomas Nikolaj Sager, Smorum (DK); Ask Püschl, Frederiksberg C (DK); Lars Torup, Vaerlose (DK)

(73) Assignee: H. LUNDBECK A/S, Valby-Copenhagen (DK)

(21) Appl. No.: 13/202,590

(22) PCT Filed: Feb. 26, 2010
(86) PCT No.: PCT/DK10/50050

§ 371 Date: Dec. 15, 2011

Related U.S. Application Data
(60) Provisional application No. 61/155,957, filed on Feb. 27, 2009.
(60) Provisional application No. 61/155,942, filed on Feb. 27, 2009.
(60) Provisional application No. 61/155,933, filed on Feb. 27, 2009.

(30) Foreign Application Priority Data
Feb. 27, 2009 (DK) PA 200900274
Feb. 27, 2009 (DK) PA 200900279
Feb. 27, 2009 (DK) PA 200900282

(51) Int. Cl. A61K 31/473; A61P 25/16

(52) U.S. Cl. 514/290

(57) ABSTRACT

Disclosed are pharmaceutical compositions and methods for the administration of (4aR,10aR)-1-n-propyl-1,2,3,4,4a,5,10,10a-octahydro-benzo[g]quinoline-6,7-diol or a pharmaceutically acceptable salt thereof and related compounds for the treatment of neurological disorder such as Parkinson's disease and restless leg syndrome.

### FIELD OF THE INVENTION

The present invention relates to methods of administering (4aR,10aR)-1-n-propyl-1,2,3,4,4a,5,10,10a-octahydro-benzo[g]quinoline-6,7-diol for the treatment of neurological disorders and pharmaceutical compositions thereof.

### BACKGROUND ART

The use of dopamine-replacing agents in the symptomatic treatment of Parkinson's disease (PD) has undoubtedly been successful in increasing the quality of life of patients. L-DOPA, which has been used for many years and remains the gold standard for treatment of PD, alleviates motor symptoms of PD characterized by the slowness of movement (bradykinesia), rigidity and/or tremor. It is understood that L-DOPA acts as a prodrug which is bio-metabolized into dopamine (DA). DA in turn activates dopamine receptors in the brain which fall into two classes: D1 and D2 receptors. D1 receptors can be divided into D₁ and D₅ receptors while D2 receptors can be divided into D₂, D₃, and D₄ receptors. However, dopamine-replacement therapy does have limitations, especially following long-term treatment.

PD afflicted patients may cycle between “on” periods in which normal functioning is attained and “off” periods in which they are severely parkinsonian. Additionally, as a consequence they may experience profound disability despite the fact that L-DOPA remains an effective anti-Parkinson agent throughout the course of the disease (Obeso, J A, et al. Neurology 2000, 55, S13-23). It is worth noting that DA agonists do cause less dyskinesia than L-DOPA but this is of limited value to PD patients with dyskinesias because many of them have moderate-to-severe PD and often they need the efficacy of L-DOPA.

Anti-Parkinson agents that mimic the action of DA have been shown to be effective in treating PD. Selective D2-agonists such as Pramipexole are effective but lack efficacy in late PD and eventually need complementation or replacement with L-DOPA. Apomorphine is a catecholamine anti-Parkinson's agent that acts as a potent D1/D2 agonist. In particular, this drug is useful as a rescue during the “off” periods of severely disabled patients who have received chronic L-DOPA treatment. However, due to its poor oral bioavailability and high first-pass effect, apomorphine is limited in its clinical application. To overcome the high first pass effect and poor oral bioavailability, apomorphine must be administered subcutaneously. Generally, the poor oral bio availability of catecholamines has prevented their clinical use as orally administered drugs.

Apart from PD, other diseases in which an increase in dopaminergic turnover may be beneficial include treating depression and for the improvement of mental functions including various aspects of cognition. Dopaminergic turnover can have a positive effect on the treatment of obesity as an anorectic agent. It can improve minimal brain dysfunction (MBD), narcolepsy, and potentially the negative, the positive as well as the cognitive symptoms of schizophrenia. Restless leg syndrome (RLS) and periodic limb movement disorder (PLMD) are alternative indications, which are clinically treated with DA agonists.

In addition, impotence and erectile dysfunction are also likely to be improved by treatment with DA agonists. Thus, improvement of sexual functions in both women and men is another possible indication for treatment with DA agonists since erectile dysfunction (impotence in men) and sexual stimulation in e.g. menopausal women (stimulation of vaginal lubrication and erection of clitoris) potentially can be achieved via DA receptor stimulation. In this context, it is noteworthy that apomorphine when given sublingually is used clinically to improve erectile dysfunction.

Clinical studies of L-DOPA and the D2 agonist Pramipexole as therapies in Huntington's disease have shown promising results; thus treatment of Huntington's disease is another potential application of the compounds of the invention. DA is involved in regulation of the cardiovascular and renal systems, and accordingly, renal failure and hypertension can be considered alternative indications for the compounds of the invention.

Despite the long-standing interest in the field, there is evidently an unmet need for developing efficient and active drugs for the treatment of PD. A mixed D1/D2 agonist giving continuous dopaminergic stimulation may fulfil such unmet needs. To this end, (4aR,10aR)-1-n-propyl-1,2,3,4,4a,5,10,10a-octahydro-benzo[g]quinoline-6,7-diol [herein referred to as Compound 10] has been identified as a potent D1/D2 agonist which shows potential to treat PD. However, as previously mentioned, the poor oral bio availability of catecholamines has prevented their clinical use as orally administered drugs.

Alternatively, the oral mucosal delivery of drugs utilizes primarily the sublingual and buccal mucosas as absorption sites, although the whole oral cavity can be considered for both mucosal (local effect) and trans-mucosal (systemic effect) absorption of drugs. Owing to the ease of administration, the oral cavity is an attractive site for delivery of drugs. Furthermore, the oral cavity has reduced enzymatic activity as compared to the intestinal, rectal, and nasal mucosas, which may lead to an improved absorption and a reduced irritation at this site of absorption. The oral cavity is less sensitive to damage and irritation than the nasal epithelium.

The oral mucosa provides a protective coating for underlying tissues while acting as a barrier to microorganisms and as a control to the passage of substances through the oral cavity. In humans, the buccal membranes consist of keratinized and nonkeratinized striated epithelium. Many factors, including partition characteristics, degree of ionization, and molecular size, influence the transport of drugs across the membrane. However, many drugs do not pass through the buccal membranes in sufficient amounts to be useful.

In general, the sublingual route is preferred for disorders requiring acute drug delivery whereas the buccal route is often utilized in cases where a prolonged drug delivery is desirable. Furthermore, a sublingual or buccal drug formulation offers an attractive alternative for patients e.g. patients suffering from Parkinson's disease having difficulties swallowing conventional oral drug formulations such as tablets or capsules. For reviews on buccal drug delivery, see: Shojaei, J. of Pharmacy & Pharm. Sci., 1998, 1, 15; Rossi et al, Drug Discovery Today 2005, 2, 1, 59; and Pather et al. Expert Opinion on Drug Delivery 2008, 5, 531. The sublingual route usually produces a faster onset of action than traditional orally administered tablets and the portion absorbed through the sublingual blood vessels bypasses the hepatic first pass metabolic processes (Motwani et al., Clin. Pharm. 1991, 21, 83-94; and Ishikawa et al., Chem. Pharm. Bull. 2001, 49, 230-232).

Due to high buccal vascularity, buccally delivered drugs can gain direct access to the systemic circulation and are not subject to first-pass hepatic metabolism. In addition, therapeutic agents administered via the buccal route are not exposed to the environment of the gastrointestinal tract (Mitra et al., Encyclopedia of Pharm. Tech. 2002, 2081-2095). Further, the buccal mucosa has low enzymatic activity relative to the nasal and rectal routes. Thus, the potential for drug inactivation due to biochemical degradation is less rapid and extensive than other administration routes (de Varies et al., Crit. Rev. Ther. Drug Carr. Syst. 1999, 8, 271-303).

Since the oral mucosa is renewed relatively fast, discoloration of the oral cavity is minimized with buccal delivery as compared to other modes of delivery. Buccal delivery is also advantageous over other modes of delivery. For example, local skin irritations are observed with the transdermal delivery of catecholamines. Further, irritation at the injection site and precipitation of decomposed apomorphine are sometimes associated with its intermittent subcutaneous administration as well as with delivery via continuous infusion.

To this end, the inventors have discovered methods to administer (4aR,10aR)-1-n-propyl-1,2,3,4,4a,5,10,10a-octahydro-benzo[g]quinoline-6,7-diol and related compounds via oral mucosa delivery. This has been achieved by the development of novel pharmaceutical compositions of said compounds for buccal administration in the treatment of Parkinson's disease as well as the other conditions disclosed in this application. Accordingly, the present invention provides pharmaceutical compositions for buccal administration comprising one of the compounds of the invention, or a pharmaceutically acceptable salt, and a pharmaceutically acceptable carrier.

Separately, the nasal mucosa offers an alternative to oral and parenteral administration; intranasal administration is a practical way to achieve the therapeutic effect of many medications. Advantages of this method are that drugs can be administered readily and simply, and either a localized or a systemic effect can be achieved. In nasal administration, the biologically active substance must be applied to the nasal mucosa in such a condition that it is able to penetrate or be absorbed through the mucosa. The extensive network of blood capillaries under the nasal mucosa is particularly suited to provide a rapid and effective systemic absorption of drugs. Moreover, the nasal epithelial membrane consists of practically a single layer of epithelial cells (pseudostratified epithelium) and may be more suited for drug administration than other mucosal surfaces having squamous epithelial layers, such as the mouth, vagina, etc.

Further, the intranasal administration of drugs that exert their effect in the brain may have the advantage in that the blood-brain-barrier (BBB) may be a less of a hurdle for the drug than if the drug had to traverse the BBB through the ‘normal’ blood stream. The onset of action may also be significantly faster for the intranasal administration of CNS based drugs than by other routes of administration.

The inventors have discovered methods to administer (4aR,10aR)-1-n-propyl-1,2,3,4,4a,5,10,10a-octahydro-benzo[g]quinoline-6,7-diol and related compounds via intranasal administration. This has been achieved by the development of novel pharmaceutical compositions of said compounds for intranasal administration in the treatment of Parkinson's disease as well as the other conditions disclosed in this application. Accordingly, the present invention provides pharmaceutical compositions for intranasal administration comprising one of the compounds of the invention, or a pharmaceutically acceptable salt, and a pharmaceutically acceptable carrier.

Moreover, delivering pharmaceutical agents into the systemic circulation through the skin is seen as a desirable route of administration while providing several other advantages over oral administration. For example, bypassing the gastrointestinal (GI) tract would obviate the GI irritation that frequently occurs and avoid partial first-pass inactivation by the liver. Further, steady absorption of drug over hours or days can be preferable to the blood level spikes and troughs produced by oral dosage forms. Additionally, patients often forget to take their medicine and even the most faithfully compliant get tired of swallowing pills, especially if they must take several each day. The transdermal route can also be more effective than the oral route in that it can provide for relatively faster or slower (extended) absorption and onset of therapeutic action.

Transdermal delivery also poses inherent challenges, in part because of the nature of skin. Skin is essentially a thick membrane that protects the body by acting as a barrier. Consequently, the movement of drugs or any external agent through the skin is a complex process. The structure of skin includes the relatively thin epidermis, or outer layer, and a thicker inner layer called the dermis. For a drug to penetrate unbroken skin, it must first move into and through the stratum corneum, which is the outer layer of the epidermis. Then the drug must penetrate the viable epidermis, papillary dermis, and capillary walls to enter the blood stream or lymph channels. Each tissue features a different resistance to penetration, but the stratum corneum is the strongest barrier to the absorption of transdermal and topical drugs. The tightly packed cells of the stratum corneum are filled with keratin. The keratinization and density of the cells may be responsible for skin's impermeability to certain drugs.

In recent years, advances in transdermal delivery include the formulation of permeation enhancers (skin penetration enhancing agents). Permeation enhancers often are lipophilic chemicals that readily move into the stratum corneum and enhance the movement of drugs through the skin. Non-chemical modes also have emerged to improve transdermal delivery; these include ultrasound, iontophoresis, and electroporation.

The inventors have discovered methods to administer (4aR,10aR)-1-n-propyl-1,2,3,4,4a,5,10,10a-octahydro-benzo[g]quinoline-6,7-diol and related compounds via transdermal delivery. This has been achieved by the development of novel pharmaceutical compositions of said compounds for transdermal administration in the treatment of Parkinson's disease as well as the other conditions disclosed in this application. Accordingly, the present invention provides pharmaceutical compositions for transdermal administration comprising one of the compounds of the invention, or a pharmaceutically acceptable salt, and a pharmaceutically acceptable carrier.

### SUMMARY OF THE INVENTION

The present invention relates a pharmaceutical composition for delivery across the oral mucosa, nasal mucosa or skin comprising (4aR,10aR)-1-n-propyl-1,2,3,4,4a,5,10,10a-octahydro-benzo[g]quinoline-6,7-diol or a pharmaceutically acceptable salt thereof, and a pharmaceutically acceptable carrier.

Another aspect relates to a use of a pharmaceutical composition for delivery across the oral mucosa, nasal mucosa or skin comprising (4aR,10aR)-1-n-propyl-1,2,3,4,4a,5,10,10a-octahydro-benzo[g]quinoline-6,7-diol or a pharmaceutically acceptable salt thereof, in the preparation of a medicament for the treatment of Parkinson's disease.

Further, aspects of the present invention relate to a pharmaceutical composition for delivery across the oral mucosa comprising (4aR,10aR)-1-n-propyl-1,2,3,4,4a,5,10,10a-octahydro-benzo[g]quinoline-6,7-diol or a pharmaceutically acceptable salt thereof, and a pharmaceutically acceptable carrier. A separate aspect is directed to a pharmaceutical composition for delivery across the oral mucosa comprising racemic trans-1-n-propyl-1,2,3,4,4a,5,10,10a-octahydro-benzo[g]quinoline-6,7-diol or a pharmaceutically acceptable salt thereof, and a pharmaceutically acceptable carrier.

Another aspect relates to a method for the delivery across the oral mucosa of the (4aR,10aR) enantiomer or the racemic trans isomer of 1-n-propyl-1,2,3,4,4a,5,10,10a-octahydro-benzo[g]quinoline-6,7-diol or a pharmaceutically acceptable salt thereof. Separately, an aspect of the invention relates to the use of a pharmaceutical composition for delivery across the oral mucosa comprising a therapeutically effective amount of the (4aR,10aR) enantiomer or the racemic trans isomer of 1-n-propyl-1,2,3,4,4a,5,10,10a-octahydro-benzo[g]quinoline-6,7-diol or a pharmaceutically acceptable salt thereof, in the preparation of a medicament for treating a neurological disorder. In one aspect, the neurological disorder is Parkinson's disease.

A separate concern of the invention is directed to a method of treating a neurological disorder comprising administering a pharmaceutical composition for delivery across the oral mucosa of a therapeutically effective amount of the (4aR,10aR) enantiomer or the racemic trans isomer of 1-n-propyl-1,2,3,4,4a,5,10,10a-octahydro-benzo[g]quinoline-6,7-diol or a pharmaceutically acceptable salt thereof. In one aspect, the neurological disorder is Parkinson's disease.

Yet another aspect of the present invention relates to a pharmaceutical composition for intranasal administration comprising (4aR,10aR)-1-n-propyl-1,2,3,4,4a,5,10,10a-octahydro-benzo[g]quinoline-6,7-diol or a pharmaceutically acceptable salt thereof, and a pharmaceutically acceptable carrier. A separate aspect is directed to a pharmaceutical composition for intranasal administration comprising racemic trans-1-n-propyl-1,2,3,4,4a,5,10,10a-octahydro-benzo[g]quinoline-6,7-diol or a pharmaceutically acceptable salt thereof, and a pharmaceutically acceptable carrier.

Another aspect relates to a method for the intranasal delivery of the (4aR,10aR) enantiomer or the racemic trans isomer of 1-n-propyl-1,2,3,4,4a,5,10,10a-octahydro-benzo[g]quinoline-6,7-diol or a pharmaceutically acceptable salt thereof. Separately, an aspect of the invention relates to the use of a pharmaceutical composition for intranasal administration comprising a therapeutically effective amount of the (4aR,10aR) enantiomer or racemic trans isomer of 1-n-propyl-1,2,3,4,4a,5,10,10a-octahydro-benzo[g]quinoline-6,7-diol or a pharmaceutically acceptable salt thereof, in the preparation of a medicament for treating a neurological disorder. In one aspect, the neurological disorder is Parkinson's disease.

A separate concern of the invention is directed to a method of treating a neurological disorder comprising administering a pharmaceutical composition for intranasal administration of a therapeutically effective amount of the (4aR,10aR) enantiomer or the racemic trans isomer of 1-n-propyl-1,2,3,4,4a,5,10,10a-octahydro-benzo[g]quinoline-6,7-diol or a pharmaceutically acceptable salt thereof. In one aspect, the neurological disorder is Parkinson's disease.

One aspect of the present invention relates to a pharmaceutical composition for transdermal delivery comprising (4aR,10aR)-1-n-propyl-1,2,3,4,4a,5,10,10a-octahydro-benzo[g]quinoline-6,7-diol or a pharmaceutically acceptable salt thereof, and a pharmaceutically acceptable carrier. A separate aspect is directed to a pharmaceutical composition for transdermal delivery comprising racemic trans-1-n-propyl-1,2,3,4,4a,5,10,10a-octahydro-benzo[g]quinoline-6,7-diol or a pharmaceutically acceptable salt thereof, and a pharmaceutically acceptable carrier.

Another aspect relates to a method for a pharmaceutical composition for transdermal delivery comprising the (4aR,10aR) enantiomer or the racemic trans isomer of 1-n-propyl-1,2,3,4,4a,5,10,10a-octahydro-benzo[g]quinoline-6,7-diol or a pharmaceutically acceptable salt thereof. Separately, an aspect of the invention relates to the use of a pharmaceutical composition for transdermal delivery comprising a therapeutically effective amount of the (4aR,10aR) enantiomer or the racemic trans isomer of 1-n-propyl-1,2,3,4,4a,5,10,10a-octahydro-benzo[g]quinoline-6,7-diol or a pharmaceutically acceptable salt thereof, in the preparation of a medicament for treating a neurological disorder. In one aspect, the neurological disorder is Parkinson's disease.

A separate concern of the invention relates to a method of treating a neurological disorder comprising administering a pharmaceutical composition for transdermal delivery of a therapeutically effective amount of the (4aR,10aR) enantiomer or the racemic trans isomer of 1-n-propyl-1,2,3,4,4a,5,10,10a-octahydro-benzo[g]quinoline-6,7-diol or a pharmaceutically acceptable salt thereof. In one aspect, the neurological disorder is Parkinson's disease.

Yet another aspect relates to a pharmaceutical composition for delivery across the oral mucosa, nasal mucosa or skin comprising a compound selected from Formula 1a, 1b or 1c:

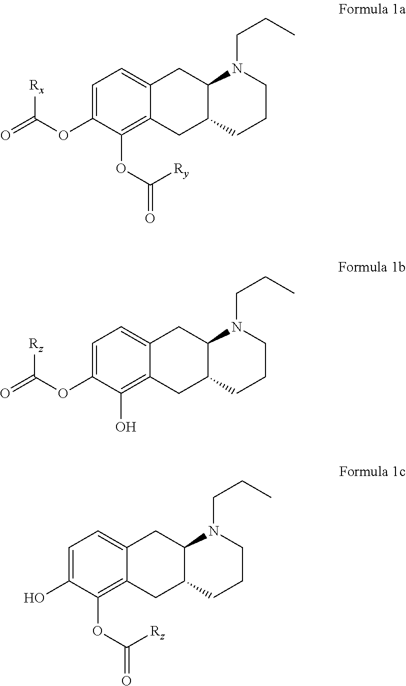

wherein each Rₓ, R_{y}, and R_{z} is independently C₁₋₆ alkanoyl, cycloalkylalkyl, phenylacetyl or benzoyl, or a pharmaceutically acceptable salt thereof, and a pharmaceutically acceptable carrier.

One aspect of the invention is directed to a ratio from about 0:1 to about 1:0 of a mixture of the asymmetric diesters of Formula Ia wherein Rx≠Ry. A separate aspect of the invention relates to a ratio from about 0:1 to about 1:0 of a mixture of the mono-esters of Formulas Ib and Ic.

Separate aspects of the invention are directed to the uses and methods of the pharmaceutical compositions described above for the treatment of Parkinson's disease.

### DETAILED DESCRIPTION

The compounds of the present invention contain two chiral centers (denoted with * in the below formula)

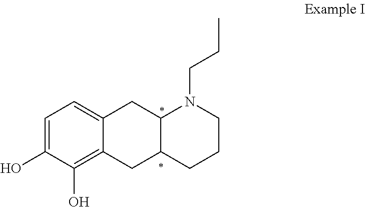

The compounds of the invention can exist in two different diastereomeric forms, the cis- and trans-isomers, both of which can exist in two enantiomeric forms. The present invention relates only to the trans racemate and the (4aR,10aR)-enantiomer.

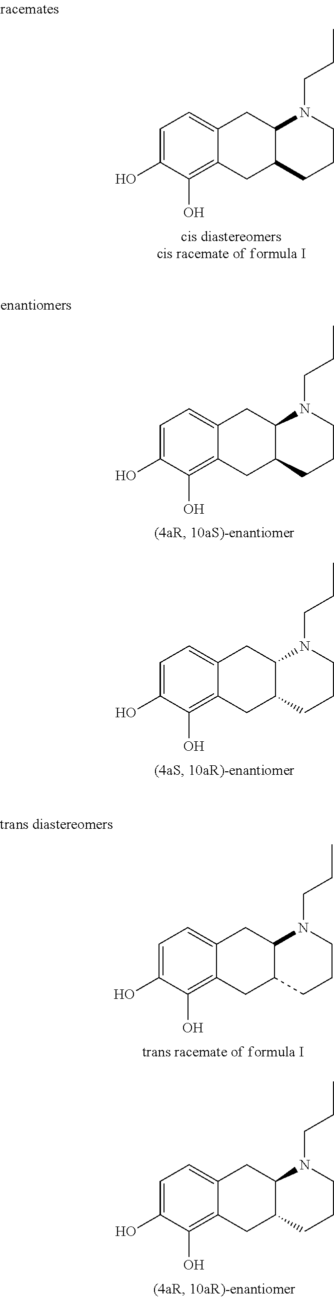

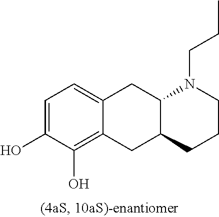

As previously indicated, the present invention is based on the discovery that (4aR,10aR)-1-n-propyl-1,2,3,4,4a,5,10,10a-octahydro-benzo[g]quinoline-6,7-diol (herein referred to as “Compound 10”) is a potent D1/D2 agonist which is bioavailable via delivery through the oral mucosa. The invention is explained in greater detail below but this description is not intended to be a detailed catalog of all the different ways in which the invention may be implemented, or all the features that may be added to the instant invention.

Racemic trans-1-n-propyl-1,2,3,4,4a,5,10,10a-octahydro-benzo[g]quinoline-6,7-diol is a 1:1 mixture of (4aR,10aR)-1-n-propyl-1,2,3,4,4a,5,10,10a-octahydro-benzo[g]quinoline-6,7-diol and (4aS,10aS)-1-n-propyl-1,2,3,4,4a,5,10,10a-octahydro-benzo[g]quinoline-6,7-diol.

“Related compounds of (4aR,10aR)-1-n-propyl-1,2,3,4,4a,5,10,10a-octahydro-benzo[g]quinoline-6,7-diol” refer to racemic trans-1-n-propyl-1,2,3,4,4a,5,10,10a-octahydro-benzo[g]quinoline-6,7-diols and the symmetric, asymmetric and mono-esters of Formulas Ia, Ib and Ic. Both the racemic trans isomer and the (4aR,10aR)-enantiomer of Formulas Ia, Ib and Ic fall within the scope of the invention.

As used herein, “C₁₋₆ alkanoyl” refers to a straight-chain or branched-chain alkanoyl group containing from one to six carbon atoms, examples of which include a formyl group, an acetyl group, a pivaloyl group, and the like.

“Cycloalkylalkyl” refers to a saturated carbocyclic ring attached to a terminal end of an a straight-chain or branched-chain alkylene linker containing one to three carbon atoms, examples of which include a cyclopropylmethyl group, a cyclobutylethyl group, a cyclopentylpropyl group, and the like.

As used herein, “active ingredient” or the “compound of the invention” refers to a compound selected from the group consisting of (4aR,10aR)-1-n-propyl-1,2,3,4,4a,5,10,10a-octahydro-benzo[g]quinoline-6,7-diol; racemic trans 1-n-propyl-1,2,3,4,4a,5,10,10a-octahydro-benzo[g]quinoline-6,7-diol; or a compound of Formulas Ia, Ib or Ic. Both the racemic trans isomer and the (4aR,10aR)-enantiomer of Formulas Ia, Ib and Ic fall within the scope of the invention.

### A. Administration Across the Oral Mucosa

As used herein, the “oral mucosal” membranes of the buccal cavity encompass the following five regions: the buccal mucosa (cheeks), the floor of the mouth (sublingual), the gums (gingiva), the palatal mucosa, and the lining of the lips.

The pharmaceutical compositions described herein may contain permeation enhancers because the buccal cavity is a poor absorptive site of the alimentary tract. The buccal cavity lacks the typical villus-type of absorptive membrane of the intestine. Further, unlike the intestine, the junction between epithelial cells are tight. For a substance to be absorbed through the mucosal membrane of the buccal cavity, it should be presented in a lipophilic form.

The delivery systems in accordance with the present invention may be used in conjunction with permeation/absorption enhancers known in the art. Suitable examples include: anionic surfactants (e.g. sodium lauryl sulfate, sodium laureate); cationic surfactants (e.g. cetylpyridinium chloride); nonionic surfactants (e.g. Polysorbate-80); bile salts (e.g. sodium glycodeoxycholate, sodium glycocholate, sodium taurodeoxycholate, sodium taurocholate); Polysaccharides (e.g. Chitosan); Synthetic polymers (e.g. Carbopol, Carbomer); Fatty acids (e.g. Oleic acid, Caprylic acid); Chelators (e.g. =Ethylenediaminetetraacetic acid, Sodium citrate); and Cyclodextrins: α, β, γ cyclodextrins. For a general review and insights on mechanism of action of absorption (permeation) enhancers for buccal application such as increasing the fluidity of the cell membrane, extracting inter/intracellular lipids, altering cellular proteins or altering surface mucin it is referred to Senel, J. Control. Res., 2001, 72:133-144.

### Antioxidants

The buccal compositions can also include one or more antioxidants. Representative antioxidants include quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, phenyl ethyl alcohol; organic acids or salts thereof such as ascorbic acid, benzoic acid, sodium benzoate, sodium ascorbate, potassium sorbate, parabens; or complex forming agents such as EDTA.

### Other Excipients

The carriers and excipients include ion-exchange microspheres which carry suitable anionic groups such as carboxylic acid residues, carboxymethyl groups, sulphopropyl groups and methylsulphonate groups. Ion-exchange resins, such as cation exchangers, can also be used. Chitosan, which is partially deacetylated chitin, or poly-N-acetyl-D-glucosamine, or a pharmaceutically acceptable salt thereof such as hydrochloride, lactate, glutamate, maleate, acetate, formate, propionate, maleate, malonate, adipate, or succinate. Suitable other ingredients for use as non-ion-exchange microspheres include starch, gelatin, collagen and albumin.

### pH Adjustment

Excipients to adjust the tonicity of the composition may be added such as sodium chloride, glucose, dextrose, mannitol, sorbitol, lactose, and the like. Acidic or basic buffers can also be added to the oral mucosal composition to control the pH. Low pH may be preferable in the instant case.

The compound of the invention as a pharmaceutical composition, may be administered in any suitable way in the oral cavity, and the compound may be presented in any suitable dosage form for such administration, e.g. in form of simple solutions or dispersions, simple tablets, matrix tablets, capsules, powders, syrups, dissolvable films, patches, lipophilic gels. In one embodiment, the compound of the invention is administered in the form of a solid pharmaceutical entity, suitably as a tablet or a capsule. In another particular embodiment, the compound of the invention is administered in the form of a dissolvable film.

In the case of oral mucosal administration of the compound of the invention, conventional dosage forms may not be able to assure therapeutic drug levels in because of physiological removal mechanism of the oral cavity (washing effect of saliva and mechanical stress), which remove the drug formulation away from the oral mucosa, resulting in too short exposure time and unpredictable absorption. To obtain the desired therapeutic action it may therefore be necessary to prolong and improve the contact between the compound of the invention and the mucosa. To fulfill the therapeutic requirement, formulations designed for sublingual or buccal administration may therefore contain mucoadhesive agents to maintain an intimate and prolonged contact of the formulation with the absorption site; penetration enhancers, to improve drug permeation across the mucosa; and enzyme inhibitors to eventually protect the drug from degradation by means of oral mucosal enzymes.

In one embodiment, the delivery across the oral mucosa occurs through buccal route. In another embodiment, the delivery across the oral mucosa occurs through the sublingual route. In another embodiment, the delivery across the oral mucosa occurs through the lips. In one embodiment, the pharmaceutical composition is a liquid solution. In one embodiment, the pharmaceutical composition is a gel. In yet another embodiment, the composition further comprises a penetration enhancer. In yet another embodiment, the composition is a tablet. In yet another embodiment, the composition is a lozenge. In yet another embodiment, the composition is a chewing gum. In yet another embodiment, the composition is a lipstick.

Methods for the preparation of solid pharmaceutical compositions are also well known in the art. Tablets may thus be prepared by mixing the active ingredient with ordinary adjuvants, fillers and diluents and subsequently compressing the mixture in a convenient tabletting machine. Examples of adjuvants, fillers and diluents comprise microcrystalline cellulose, corn starch, potato starch, lactose, mannitol, sorbitol talcum, magnesium stearate, gelatine, lactose, gums, and the like. Any other adjuvant or additive such as colorings, aroma, preservatives, etc. may also be used provided that they are compatible with the active ingredients.

In particular, the tablet formulations according to the invention may be prepared by direct compression of the compound of the invention with conventional adjuvants or diluents. Alternatively, a wet granulate or a melt granulate of the compound of the invention, optionally in admixture with conventional adjuvants or diluents may be used for compression of tablets.

In a specific embodiment of the invention there is provided a pharmaceutical composition comprising a therapeutically effective amount of the compound of the invention, or a pharmaceutically acceptable acid addition salt thereof for administration via the oral mucosa, in particular buccally or sublingually.

Manufacturing processes for buccal and sublingual disintegrating tablets are known in the art and include, but are not limited to, conventional tableting techniques, freeze-dried technology, and floss-based tableting technology.

### Conventional Tableting Techniques

Conventional tablet processing features conventional tablet characteristics for ease of handling, packaging, and fast disintegration (Ghosh and Pfister, Drug Delivery to the Oral Cavity: Molecule to Market, 2005, New York, CRC Press). The technology is based on a combination of physically modified polysaccharides that have water dissolution characteristics that facilitate fast disintegration and high compressibility. The result is a fast-disintegrating tablet that has adequate hardness for packaging in bottles and easy handling.

In certain embodiments, the manufacturing process involves granulating low-moldable sugars (e.g., mannitol, lactose, glucose, sucrose, and erythritol) that show quick dissolution characteristics with high-moldable sugars (e.g., maltose, sorbitol, trehalose, and maltitol). The result is a mixture of excipients that have fast-dissolving and highly moldable characteristics (Hamilton et al., Drug Deliv. Technol. 2005, 5, 34-37). The compound of the invention can be added, along with other standard tableting excipients, during the granulation or blending processes. The tablets are manufactured at a low compression force followed by an optional humidity conditioning treatment to increase tablet hardness (Parakh et al., Pharm. Tech. 2003, 27, 92-100).

In other embodiments, a compressed buccal or sublingual tablet comprising the compound of the invention is based on a conventional tableting process involving the direct compression of active ingredients, effervescent excipients, and taste-masking agents (see U.S. Pat. No. 5,223,614). The tablet quickly disintegrates because effervescent carbon dioxide is produced upon contact with moisture. The effervescent excipient (known as effervescence couple) is prepared by coating the organic acid crystals using a stoichiometrically lesser amount of base material. The particle size of the organic acid crystals is carefully chosen to be larger than the base excipient to ensure uniform coating of the base excipient onto the acid crystals. The coating process is initiated by the addition of a reaction initiator, which is purified water in this case. The reaction is allowed to proceed only to the extent of completing the base coating on organic acid crystals. The required end-point for reaction termination is determined by measuring carbon dioxide evolution. Then, the excipient is mixed with the active ingredient or active microparticles and with other standard tableting excipients and then compressed into tablets.

In still other embodiments, the buccal or sublingual tablets are made by combining non-compressible fillers with a taste-masking excipient and active ingredient into a dry blend. The blend is compressed into tablets using a conventional rotary tablet press. Tablets made with this process have higher mechanical strength and are sufficiently robust to be packaged in blister packs or bottles (Aurora et al., Drug Deliv. Technol. 2005, 5:50-54). In other embodiments, the method further incorporates taste-masking sweeteners and flavoring agents such as mint, cherry, and orange. In certain embodiments, the compound of the invention tablets made with this process should disintegrate in the mouth in 5-45 seconds and can be formulated to be bio equivalent to intramuscular or subcutaneous dosage forms containing the compound of the invention.

### Freeze-Dried Buccal or Sublingual Tablets

The freeze-drying process involves the removal of water (by sublimation upon freeze drying) from the liquid mixture of the compound of the invention matrix former, and other excipients filled into preformed blister pockets. The formed matrix structure is very porous in nature and rapidly dissolves or disintegrates upon contact with saliva (Sastry et al., Drug Delivery to the Oral Cavity Molecule to Market, 2005, New York, CRC Press, pp. 311-316).

Common matrix-forming agents include gelatins, dextrans, or alginates which form glassy amorphous mixtures for providing structural strength; saccharides such as mannitol or sorbitol for imparting crystallinity and hardness; and water, which functions as a manufacturing process medium during the freeze-drying step to induce the porous structure upon sublimation. In addition, the matrix may contain taste-masking agents such as sweeteners, flavorants, pH-adjusting agents such as citric acid, and preservatives to ensure the aqueous stability of the suspended drug in media before sublimation.

In this embodiment, freeze-dried buccal or sublingual Oral Disintegrating Tablets (herein referred to as ODTs) comprising the compound of the invention can be manufactured and packaged in polyvinyl chloride or polyvinylidene chloride plastic packs, or they may be packed into laminates or aluminum multilaminate foil pouches to protect the product from external moisture.

Other known methods for manufacturing buccal or sublingual ODTs include lyophilization (e.g., Lyoc (Farmalyoc, now Cephalon, Franzer, Pa.) and QuickSolv (Janssen Pharmaceutica, Beerse, Belgium). Lyoc is a porous, solid wafer manufactured by lyophilizing an oil-in-water emulsion placed directly in a blister and subsequently sealed. The wafer can accommodate high drug dosing and disintegrates rapidly but has poor mechanical strength (see EP 0159237). QuickSolv tablets are made with a similar technology that creates a porous solid matrix by freezing an aqueous dispersion or solution of the matrix formulation. The process works by removing water using an excess of alcohol (solvent extraction). In certain embodiments, the manufacturing methods which utilize the lyophilization techniques, such as those related to QuickSolv as described above, could be of particular importance for producing buccal or sublingual ODTs comprising the compound of the invention. This is especially so in light of the data provided herein which shows the potential negative effect that highly water soluble excipients can have in the absorption of the compound of the invention in vivo. Thus, a buccal or sublingual ODT comprising the compound of the invention manufactured by such a lyophilization technique could provide increased in vivo absorption due of the removal of water soluble excipients occurring during the water removal step as described above.

### Floss-Based Buccal or Sublingual Tablets

In other embodiments, floss-based tablet technology (e.g., FlashDose, Biovail, Mississauga, ON, Canada) can be used to produce fast-dissolving buccal or sublingual tablets comprising the compound of the invention using a floss known as the shearform matrix. This floss is commonly composed of saccharides such as sucrose, dextrose, lactose, and fructose. The saccharides are converted into floss by the simultaneous action of flash-melting and centrifugal force in a heat-processing machine similar to that used to make cotton candy. See U.S. Pat. Nos. 5,587,172, 5,622,717, 5,567,439, 5,871,781, 5,654,003, and 5,622,716. The fibers produced are usually amorphous in nature and are partially re-crystallized, which results in a free-flowing floss. The floss can be mixed with the compound of the invention and pharmaceutically acceptable excipients followed by compression into a tablet that has fast-dissolving characteristics.

### Sublingual Tablets

Additional techniques can also be used to formulate the rapidly disintegrating or dissolving buccal or sublingual tablets of the present invention (Sastry et al., Pharm. Sci. Tech. Today 2000, 3: 138-145; Chang et al., Pharmaceutical Technology 2000, 24: 52-58; Sharma et al., Pharmaceutical Technology North America 2003, 10-15; Allen, International Journal of Pharmaceutical Technology 2003, 7, 449-450; Dobetti, Pharmaceutical Technology Europe 2000, 12: 32-42; and Verma and Garg, Pharmaceutical Technology On-Line 2001, 25, 1-14). Direct compression, one of these techniques, requires the incorporation of a super disintegrant into the formulation, or the use of highly water soluble excipients to achieve fast tablet disintegration or dissolution. Direct compression does not require the use of moisture or heat during tablet formation process, so it is very useful for the formulation and compression of tablets containing moisture-labile and heat-labile medications. However, the direct compression method is very sensitive to changes in the types and proportions of excipients, and in the compression force (CF), when used to achieve tablets of suitable hardness without compromising the rapid disintegration capabilities. As will be appreciated by one of skill in the art, in order for tablets administered sublingually to release the dose of medication for maximum rate and extent of absorption, the tablet must disintegrate almost instantaneously following insertion into the sublingual cavity. Precise selection and evaluation of the type and proportion of excipients used to formulate the tablet control the extent of hardness and rate of disintegration. Compression force (CF) can also be adjusted to result in tablets that have lower hardness (H) and disintegrate more quickly. Unique packaging methods such as strip packaging may be required to compensate for the problem of extreme friability of rapidly disintegrating, direct compression tablets.

Watenabe et al. (Watanabe et al., Biol. Pharm. Bull. 1995, 18: 1308-1310; Ishikawa et al., Chem. Pharm. Bull. 2001, 49: 134-139) and Bi et al (Bi et al., Chem. Pharm. Bull. 1996, 44: 2121-2127; Bi et al., Drug Dev. Lnd. Pharm. 1999, 25: 571-581) were the first to evaluate the ideal excipient proportions and other related parameters required to formulate durable fast disintegrating tablets using a super disintegrant. They studied the effect of a wide range of microcrystalline cellulose: low-substituted hydroxypropyl cellulose (MCC:L HPC) ratios on the tablet characteristics.

In a further aspect the invention provides the use of said composition for the preparation of a medicament for the treatment of neurodegenerative disorders such as Parkinson's disease and Huntington's disease.

In a further aspect the invention provides the use of the pharmaceutical composition for the preparation of a medicament for the treatment of psychoses, impotence, renal failure, heart failure or hypertension.

In another aspect the invention provides the use of the pharmaceutical composition for the manufacture of a medicament for the treatment of cognitive impairment in a mammal.

In a still further aspect the invention provides the use of the pharmaceutical composition for the manufacture of a medicament for the treatment of restless legs syndrome (RLS) or periodic limb movement disorder (PLMD).

In a still further aspect the invention provides the use of the pharmaceutical composition for the manufacture of a medicament for the treatment of erectile dysfunction.

In a different aspect the invention provides the use of the pharmaceutical composition for the manufacture of a medicament for the treatment of movement disorders, poverty of movement, dyskinetic disorders, gait disorders or intention tremor in a mammal.

In a further aspect the invention provides the use of the pharmaceutical composition for the treatment of neurodegenerative disorders such as Parkinson's disease and Huntington's disease.

In a further aspect the invention provides the use of the pharmaceutical composition for the treatment of psychoses, impotence, renal failure, heart failure or hypertension.

In another aspect the invention provides the use of the pharmaceutical composition for the treatment of cognitive impairment in a mammal.

In a still further aspect the invention provides the use of the pharmaceutical composition for the treatment of restless legs syndrome (RLS) or periodic limb movement disorder (PLMD).

In a different aspect the invention provides the use of the pharmaceutical composition for the treatment of movement disorders, poverty of movement, dyskinetic disorders, gait disorders or intention tremor in a mammal.

In separate aspects the invention provides the use of the pharmaceutical composition for the manufacture of medicaments, which are intended for administration via the oral mucosa.

The invention also provides a method of treating a mammal suffering from a neurodegenerative disorder such as Parkinson's disease and Huntington's disease comprising administering to the mammal a therapeutically effective amount of the pharmaceutical composition.

In another aspect the invention also provides a method of treating a mammal suffering from psychoses, impotence, renal failure, heart failure or hypertension, comprising administering to the mammal a therapeutically effective amount of the pharmaceutical composition.

In a further aspect the invention provides a method of treating a mammal suffering from a cognitive impairment, comprising administering to the mammal an effective amount of the pharmaceutical composition.

The invention also relates to a method of treating a mammal suffering from restless legs syndrome (RLS) or periodic limb movement disorder (PLMD), comprising administering to the mammal a therapeutically effective amount of the compound of the invention, or a pharmaceutically acceptable addition salt thereof.

The invention also relates in a separate aspect to a method of treating a mammal suffering from movement disorders, poverty of movement, dyskinetic disorders, gait disorders or intention tremor comprising administering to the mammal of the pharmaceutical composition.

The therapeutically effective amount of the compound of the invention, calculated as the daily dose of the compound of the invention above as the free base, is suitably between 0.001 and 12.5 mg/day, more suitable between 0.005 and 10.0 mg/day, e.g. preferably between 0.01 and 5.0 mg/day. In a specific embodiment the daily dose of the compound of the invention is between 0.1 and 1.0 mg/day.

In another embodiment the daily dose of the compound of the invention is less than about 0.1 mg/day. In a separate embodiment the daily dose of the compound of the invention is about 0.01 mg/day. In a further embodiment the invention provides a formulation comprising from 0.0001 mg to 12.5 mg of the compound of the invention for delivery via the oral mucosa. In a further embodiment the invention provides a formulation comprising from 0.0001 mg to 0.01 mg of the compound of the invention for delivery via the oral mucosa. In a further embodiment the invention provides a formulation comprising from 0.001 mg to 0.10 mg of the compound of the invention for delivery via the oral mucosa. In a further embodiment the invention provides a formulation comprising from 0.01 mg to 1.0 mg of the compound of the invention for delivery via the oral mucosa.

In yet other embodiments, the invention described herein provides pharmaceutical tablets for buccal or sublingual administration comprising the compound of the invention wherein the administration of the pharmaceutical tablets provides a pharmacokinetic profile substantially equivalent to the pharmacokinetic profile of traditional injectable dosage forms comprising the compound of the invention administered either subcutaneously or intramuscularly. In certain embodiments, the pharmaceutical tablets for buccal or sublingual administration described herein can provide a pharmacokinetic profile substantially equivalent to the pharmacokinetic profile of traditional injectable dosage forms comprising the compound of the invention administered either subcutaneously or intramuscularly, wherein the pharmacokinetic profile consists of one or more of the pharmacokinetic parameters selected from the group consisting of: Cₘₐₓ, T_{maχ}, AUC₍ₗₐₛₜ₎, and AUC_{(0-∞)}.

Ultimately, the exact dose of the compound of the invention and the particular formulation to be administered depend on a number of factors, e.g., the condition to be treated, the desired duration of the treatment and the rate of release of the active agent. For example, the amount of the active agent required and the release rate thereof may be determined on the basis of known in vitro or in vivo techniques, determining how long a particular active agent concentration in the blood plasma remains at an acceptable level for a therapeutic effect.

### B. Intranasal Administration

The term “intranasal delivery” as used herein means a method for drug absorption through and within the nasal mucosa.

Carriers” or “vehicles” as used herein refer to carrier materials suitable for intranasal drug administration, and include any such materials known in the art, e.g., any liquid, gel, solvent, liquid diluent, solubilizer, or the like, which is non toxic and which does not interact with other components of the composition in a deleterious manner. Examples of suitable vehicles for use herein include water, alcohols such as isopropyl alcohol and isobutyl alcohol, polyalcohol such as glycerol, and glycols such as propylene glycol, and esters of such polyols, (e.g., mono-, di-, or tri-glycerides).

### Intranasal Compositions

Relative to an oral dosage form such as a tablet or capsule, intranasal delivery provides for rapid absorption, faster onset of therapeutic action and avoidance of gut wall or liver first pass metabolism. For patients who have difficulty in swallowing tablets, capsules or other solids or those who have intestinal failure, the intranasal delivery route may be preferred.

The compositions for nasal administration include the compound of the invention, or a pharmaceutically acceptable salt thereof, and optionally can also include other ingredients including, but not limited to, carriers and excipients, such as absorption-promoting agents which promote nasal absorption of the active ingredient after nasal administration. Other optional excipients include diluents, binders, lubricants, glidants, disintegrants, desensitizing agents, emulsifiers, mucosal adhesives, solubilizers, suspension agents, viscosity modifiers, ionic tonicity agents, buffers, carriers, flavors and mixtures thereof.

The amount of drug absorbed depends on many factors. These factors include the drug concentration, the drug delivery vehicle, mucosal contact time, the venous drainage of the mucosal tissues, the degree that the drug is ionized at the pH of the absorption site, the size of the drug molecule, and its relative lipid solubility. Those of skill in the art can readily prepare an appropriate intranasal composition, which delivers an appropriate amount of the active agent, taking these factors into consideration.

### Absorption Promoting Agents

The transport of the active ingredient across normal nasal mucosa can be enhanced by optionally combining it with an absorption promoting agent, such as those disclosed in U.S. Pat. Nos. 5,629,011, 5,023,252, 6,200,591, 6,369,058, 6,380,175, and International Publication Number WO 01/60325. Examples of these absorption promoting agents include, but are not limited to, cationic polymers, surface active agents, chelating agents, mucolytic agents, cyclodextrin, polymeric hydrogels, combinations thereof, and any other similar absorption promoting agents known to those of skill in the art. Representative absorption promoting excipients include phospholipids, such as phosphatidylglycerol or phosphatidylcholine, lysophosphatidyl derivatives, such as lysophosphatidylethanolamine, lysophosphatidylcholine, lysophosphatidylglycerol, lysophosphatidylserine, or lysophosphatidic acid, polyols, such as glycerol or propylene glycol, fatty acid esters thereof such as glycerides, amino acids, and esters thereof, and cyclodextrins. Gelling excipients or viscosity-increasing excipients can also be used.

### Mucoadhesive/Bioadhesive Polymers

The transport of the active ingredient across normal mucosal surfaces can also be enhanced by increasing the time in which the formulations adhere to the mucosal surfaces. Mucoadhesive/bioadhesive polymers, for example, those which form hydrogels, exhibit mucoadhesion and controlled drug release properties and can be included in the intranasal compositions described herein. Examples of such formulations are disclosed in U.S. Pat. Nos. 6,068,852 and 5,814,329; and International Publication Number WO99/58110. Representative bioadhesive or hydrogel-forming polymers capable of binding to the nasal mucosa are well known to those of skill in the art, and include polycarbophil, polylysine, methylcellulose, sodium carboxymethylcellulose, hydroxypropyl-methylcellulose, hydroxyethyl cellulose, pectin, Carbopol 934P, polyethylene oxide 600K, Pluronic F127, polyisobutylene (PIB), polyisoprene (PIP), polyvinyl pyrrolidone (PVP), polyvinyl alcohol (PVA), xanthum gum, guar gum, and locust bean gum.

Other nasal delivery compositions are chitosan-based and are suitable to increase the residence time of the active ingredient on mucosal surfaces, which results in increasing its bioavailability. Examples of these nasal delivery compositions are disclosed in U.S. Pat. Nos. 6,465,626, 6,432,440, 6,391,318, and 5,840,341; European Patent Numbers EP0993483 and EP1051190; and International Publication Numbers WO 96/05810, WO 96/03142, and WO 93/15737. Additionally, the present invention can be formulated with powder microsphere and mucoadhesive compositions as disclosed in European Patent Numbers EP1025859 and EP1108423, which are incorporated herein by reference with regard to such composition.

Finally, thiolated polymeric excipients that form covalent bonds with the cysteine-rich subdomains of the mucus membrane can also provide mucoadhesion, which prolongs the contact time between the active ingredient and the membrane. Such excipients are disclosed in International Publication Number WO 03/020771.

### Antioxidants

The buccal compositions can also include one or more antioxidants. Representative antioxidants include quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, phenyl ethyl alcohol; organic acids or salts thereof such as ascorbic acid, benzoic acid, sodium benzoate, sodium ascorbate, potassium sorbate, parabens; or complex forming agents such as ethylenediaminetetraacetic acid (EDTA).

### Other Excipients

The carriers and excipients include ion-exchange microspheres which carry suitable anionic groups such as carboxylic acid residues, carboxymethyl groups, sulphopropyl groups and methylsulphonate groups. Ion-exchange resins, such as cation exchangers, can also be used. Chitosan, which is partially deacetylated chitin, or poly-N-acetyl-D-glucosamine, or a pharmaceutically acceptable salt thereof such as hydrochloride, lactate, glutamate, maleate, acetate, formate, propionate, maleate, malonate, adipate, or succinate. Suitable other ingredients for use as non-ion-exchange microspheres include starch, gelatin, collagen and albumin.

The composition can also include an appropriate acid selected from the group consisting of hydrochloric acid, lactic acid, glutamic acid, maleic acid, acetic acid, formic acid, propionic acid, malic acid, malonic acid, adipic acid, and succinic acid. Other ingredients such as diluents are cellulose, microcrystalline cellulose, hydroxypropyl cellulose, starch, hydroxypropylmethyl cellulose, and the like.

Excipients to adjust the tonicity of the composition may be added such as sodium chloride, glucose, dextrose, mannitol, sorbitol, lactose, and the like. Acidic or basic buffers can also be added to the intranasal composition to control the pH.

Incorporation of the Active Agent into the Compositions

In addition to using absorption enhancing agents, which increase the transport of the active agents through the mucosa, and bioadhesive materials, which prolong the contact time of the active agent along the mucosa, the administration of the active agent can be controlled by using controlled release formulations, which can provide rapid or sustained release, or both, depending on the formulations.

There are numerous particulate drug delivery vehicles known to those of skill in the art which can include the active ingredients, and deliver them in a controlled manner. Examples include particulate polymeric drug delivery vehicles, for example, biodegradable polymers, and particles formed of non-polymeric components. These particulate drug delivery vehicles can be in the form of powders, microparticles, nanoparticles, microcapsules, liposomes, and the like. Typically, if the active agent is in particulate form without added components, its release rate depends on the release of the active agent itself. Typically, the rate of absorption is enhanced by presenting the drug in a micronized form, wherein particles are below 20 microns in diameter. In contrast, if the active agent is in particulate form as a blend of the active agent and a polymer, the release of the active agent is controlled, at least in part, by the removal of the polymer, typically by dissolution, biodegradation, or diffusion from the polymer matrix.

The compositions can provide an initial rapid release of the active ingredient followed by a sustained release of the active ingredient. U.S. Pat. No. 5,629,011 provides examples of this type of formulation and is incorporated herein by reference with regard to such formulations.

There are numerous compositions that utilize intranasal delivery and related methods thereof. Moreover, there are numerous methods and related delivery vehicles that provide for intranasal delivery of various pharmaceutical compositions. For example, intranasal compositions that employ current marketed nicotine replacement therapies (See, N. J. Benowitz, Drugs, 45: 157-170 (1993) are also suitable for administering the compounds described herein.

### Nasal Insufflator Devices

The intranasal compositions can be administered by any appropriate method according to their form. A composition including microspheres or a powder can be administered using a nasal insufflator device. Examples of these devices are well known to those of skill in the art, and include commercial powder systems such as Fisons Lomudal System. An insufflator produces a finely divided cloud of the dry powder or microspheres. The insufflator is preferably provided with a mechanism to ensure administration of a substantially fixed amount of the composition. The powder or microspheres can be used directly with an insufflator, which is provided with a bottle or container for the powder or microspheres. Alternatively, the powder or microspheres can be filled into a capsule such as a gelatin capsule, or other single dose device adapted for nasal administration. The insufflator preferably has a mechanism to break open the capsule or other device.

Further, the composition can provide an initial rapid release of the active ingredient followed by a sustained release of the active ingredient, for example, by providing more than one type of microsphere or powder.

### Use of Metered Sprays

Intranasal delivery can also be accomplished by including the active ingredient in a solution or dispersion in an aqueous medium which can be administered as a spray. Appropriate devices for administering such a spray include metered dose aerosol valves and metered dose pumps, optionally using gas or liquid propellants.

Representative devices of this type are disclosed in the following patents, patent applications, and publications: WO 03/026559, WO 02/011800, WO 00/51672, WO 02/068029, WO 02/068030, WO 02/068031, WO 02/068032, WO 03/000310, WO 03/020350, WO 03/082393, WO 03/084591, WO 03/090812, WO 00/41755, and the pharmaceutical literature (See, Bell, A. Intranasal Delivery Devices, in Drug Delivery Devices Fundamentals and Applications, Tyle P. (ed), Dekker, New York, 1988); and Remington's Pharmaceutical Sciences, Mack Publishing Co., 1975.

### Other Modes of Intranasal Delivery

In addition to the foregoing, the compounds and intranasal compositions including the compounds can also be administered in the form of nose-drops, sprays, irrigations, and douches, as is known in the art. Nose drops are typically administered by inserting drops while lying on a bed, with the patient on his or her back, especially with the head lying over the side of the bed. This approach helps the drops get farther back.

Nasal irrigation involves regularly flooding the nasal cavity with warm salty water, which includes one or more compounds as described herein, or their pharmaceutically acceptable salts. Nasal douches are typically used by filling a nasal douche with a salt solution including one or more compounds as described herein, or their pharmaceutically acceptable salts, inserting the nozzle from the douche into one nostril, opening one's mouth to breathe, and causing the solution to flow into one nostril, rinse round the septum and turbinates, and discharge from the other nostril.

As mentioned previously, the present invention provides pharmaceutical compositions for intranasal administration of (4aR,10aR)-1-n-propyl-1,2,3,4,4a,5,10,10a-octahydro-benzo[g]quinoline-6,7-diol and related compounds, which may be delivered to the systemic circulation via delivery across the nasal mucosa.

In one embodiment, the composition is further comprising an absorption agent. In one embodiment, the composition is further comprising one or more adhesive, binder, lubricant, glidant, disintegrant or mixture thereof.

The compound of the invention as a pharmaceutical composition for intranasal administration may be administered in any suitable way in the nasal cavity, and the compound may be presented in any suitable dosage form for such administration, e.g. in form of simple solutions or dispersions, simple tablets, matrix tablets, capsules, powders, syrups, dissolvable films, patches, lipophilic gels. In one embodiment, the compound of the invention is administered in the form of a solid pharmaceutical entity, suitably as a tablet or a capsule. In another particular embodiment, the compound of the invention is administered in the form of a dissolvable film.

In the case of intranasal administration of the compound of the invention, conventional dosage forms may not be able to assure therapeutic drug levels in because of physiological removal mechanism of the oral cavity (washing effect of saliva and mechanical stress), which remove the drug formulation away from the nasal mucosa, resulting in too short exposure time and unpredictable absorption. To obtain the desired therapeutic action it may therefore be necessary to prolong and improve the contact between the compound of the invention and the nasal mucosa. To fulfill the therapeutic requirement, formulations designed for intranasal administration may therefore contain mucoadhesive agents to maintain an intimate and prolonged contact of the formulation with the absorption site; penetration enhancers, to improve drug permeation across the mucosa; and enzyme inhibitors to eventually protect the drug from degradation by means of nasal mucosal enzymes.

In a specific embodiment of the invention there is provided a pharmaceutical composition comprising a therapeutically effective amount of compound of the invention or a pharmaceutically acceptable acid addition salt thereof for administration via the nasal mucosa.

In a further aspect the invention provides the use of said composition for the preparation of a medicament for the treatment of neurodegenerative disorders such as Parkinson's disease and Huntington's disease.

In a further aspect the invention provides the use of the pharmaceutical composition for the preparation of a medicament for the treatment of psychoses, impotence, renal failure, heart failure or hypertension.

In another aspect the invention provides the use of the pharmaceutical composition for the manufacture of a medicament for the treatment of cognitive impairment in a mammal.

In a still further aspect the invention provides the use of the pharmaceutical composition for the manufacture of a medicament for the treatment of restless legs syndrome (RLS) or periodic limb movement disorder (PLMD).

In a still further aspect the invention provides the use of the pharmaceutical composition for the manufacture of a medicament for the treatment of erectile dysfunction.

In a different aspect the invention provides the use of the pharmaceutical composition for the manufacture of a medicament for the treatment of movement disorders, poverty of movement, dyskinetic disorders, gait disorders or intention tremor in a mammal.

In a further aspect the invention provides the use of the pharmaceutical composition for the treatment of neurodegenerative disorders such as Parkinson's disease and Huntington's disease.

In a further aspect the invention provides the use of the pharmaceutical composition for the treatment of psychoses, impotence, renal failure, heart failure or hypertension.

In another aspect the invention provides the use of the pharmaceutical composition for the treatment of cognitive impairment in a mammal.

In a still further aspect the invention provides the use of the pharmaceutical composition for the treatment of restless legs syndrome (RLS) or periodic limb movement disorder (PLMD).

In a different aspect the invention provides the use of the pharmaceutical composition for the treatment of movement disorders, poverty of movement, dyskinetic disorders, gait disorders or intention tremor in a mammal.

In separate aspects the invention provides the use of the pharmaceutical composition for the manufacture of medicaments, which are intended for administration via the oral mucosa.

The invention also provides a method of treating a mammal suffering from a neurodegenerative disorder such as Parkinson's disease and Huntington's disease comprising administering to the mammal a therapeutically effective amount of the pharmaceutical composition.

In another aspect the invention also provides a method of treating a mammal suffering from psychoses, impotence, renal failure, heart failure or hypertension, comprising administering to the mammal a therapeutically effective amount of the pharmaceutical composition.

In a further aspect the invention provides a method of treating a mammal suffering from a cognitive impairment, comprising administering to the mammal an effective amount of the pharmaceutical composition.

The invention also relates to a method of treating a mammal suffering from restless legs syndrome (RLS) or periodic limb movement disorder (PLMD), comprising administering to the mammal a therapeutically effective amount of compound of the invention, or a pharmaceutically acceptable addition salt thereof.

The invention also relates in a separate aspect to a method of treating a mammal suffering from movement disorders, poverty of movement, dyskinetic disorders, gait disorders or intention tremor comprising administering to the mammal of the pharmaceutical composition.

The therapeutically effective amount of the compound of the invention, calculated as the daily dose of the compound of the invention above as the free base, is suitably between 0.001 and 12.5 mg/day, more suitable between 0.005 and 10.0 mg/day, e.g. preferably between 0.01 and 5.0 mg/day. In a specific embodiment the daily dose of the compound of the invention is between 0.1 and 1.0 mg/day.

In another embodiment the daily dose of the compound of the invention is less than about 0.1 mg/day. In a separate embodiment the daily dose of the compound of the invention is about 0.01 mg/day. In a further embodiment the invention provides a formulation comprising from 0.0001 mg to 12.5 mg of the compound of the invention for delivery via the nasal mucosa. In a further embodiment the invention provides a formulation comprising from 0.0001 mg to 0.01 mg of the compound of the invention for delivery via the nasal mucosa. In a further embodiment the invention provides a formulation comprising from 0.001 mg to 0.10 mg of the compound of the invention for delivery via the nasal mucosa. In a further embodiment the invention provides a formulation comprising from 0.01 mg to 1.0 mg of the compound of the invention for delivery via the nasal mucosa.

C. Transdermal Administration By “transdermal delivery”, applicants intend to include both transdermal and percutaneous administration, i.e., delivery by passage of an active ingredient through the skin and into the bloodstream.

“Carriers” or “vehicles” as used herein refer to carrier materials suitable for transdermal drug administration, and include any such materials known in the art, e.g., any liquid, gel, solvent, liquid diluent, solubilizer, or the like, which is non toxic and which does not interact with other components of the composition in a deleterious manner. Examples of suitable vehicles for use herein include water, alcohols such as isopropyl alcohol and isobutyl alcohol, polyalcohols such as glycerol, and glycols such as propylene glycol, and esters of such polyols, (e.g., mono-, di-, or tri-glycerides).

“Penetration enhancement” or “permeation enhancement” as used herein relates to an increase in the permeability of skin to a pharmacologically active agent, namely, so as to increase the rate at which the active ingredient permeates through the skin (i.e., flux) and enters the bloodstream or the local site of action. The enhanced permeation effected by using these enhancers can be observed by measuring the rate of diffusion (or flux) of active ingredient through animal or human skin or a suitable polymeric membrane using a diffusion cell apparatus as described in the examples herein.

Permeation enhancers are described, for example, in U.S. Pat. Nos. 5,785,991; 4,764,381; 4,956,171; 4,863,970; 5,453,279; 4,883,660; 5,719,197, and in the literature “Pharmaceutical Skin Penetration Enhancement”, J. Hadgraft, Marcel Dekker, Inc. 1993; “Percutaneous Absorption”, R. Bronaugh, H. Maibach, Marcel Dekker, Inc. (1989), B. W. Barry, “Penetration Enhancers in Skin Permeation”, Proceedings of the 13th international Symposium on Controlled Release of Bioactive Materials, ed. by Chaudry & Thies, Controlled Release Society, Lincolnshire, III., pp. 136-137 (1986), and Cooper & Berner, “Penetration Enhancers”, in The Transdermal Delivery of Ingredients, Vol. Il ed. by Kydonieus and Berner, CRC Press, Boca Raton, FIa. pp. 57-62 (1986).

The permeation enhancers should both enhance the permeability of the stratum corneum, and be non-toxic, non-irritant and non-sensitizing on repeated exposure. Representative permeation enhancers include, for example, sucrose monococoate, glycerol monooleate, sucrose monolaurate, glycerol monolaureate, diethylene glycol monoalkyl ethers such as diethylene glycol monoethyl or monomethyl ether (Transcutol® P), ester components such as propylene glycol monolaurate, methyl laurate, and lauryl acetate, monoglycerides such as glycerol monolaurate, fatty alcohols such as lauryl alcohol, and 2-ethyl-1,3 hexanediol alone or in combination with oleic acid.

### Gelling Agents

Gelling agents, such as carbomer, carboxyethylene or polyacrylic acid such as Carbopol® 980 or 940 NF, 981 or 941 NF, 1382 or 1342 NF, 5984 or 934 NF, ETD 2020, 2050, 934P NF, 971 P NF, 974P NF, Noveon® AA-1 USP, etc; cellulose derivatives such as ethylcellulose, hydroxypropylmethylcellulose (HPMC), ethylhydroxyethylcellulose (EHEC), carboxymethylcellulose (CMC), hydroxypropylcellulose (HPC) (Klucel®, different grades), hydroxyethylcellulose (HEC) (Natrosol® grades), HPMCP 55, Methocel® grades, etc; natural gums such as arabic, xanthan, guar gums, alginates, etc; polyvinylpyrrolidone derivatives such as Kollidon® grades; polyoxyethylene polyoxypropylene copolymers such as Lutrol® F grades 68, 127, etc; others like chitosan, polyvinyl alcohols, pectins, veegun grades, and the like, can also be present. Those of the skill in the art know of other gelling agents or viscosants suitable for use in the present invention. Representative gelling agents include, but are not limited to, Carbopol® 980 NF, Lutrol® F 127, Lutrol® F 68 and Noveon® AA-1 USP. The gelling agent is present from about 0.2 to about 30.0% w/w, depending on the type of polymer.

### Antioxidants

The transdermal compositions can also include one or more antioxidants. Representative antioxidants include quaternary ammonium salts such as lauralkonium chloride, benzalkonium chloride, benzododecinium chloride, cetyl pyridium chloride, cetrimide, domiphen bromide; alcohols such as benzyl alcohol, chlorobutanol, o-cresol, phenylethyl alcohol; organic acids or salts thereof such as ascorbic acid, benzoic acid, sodium ascorbate, sodium benzoate, potassium sorbate, parabens; or complex forming agents such as ethylenediaminetetraacetic acid (EDTA). Representative antioxidants include butylhydroxytoluene, butylhydroxyanisole, ethylenediaminetetraacetic acid and its sodium salts, D,L-alpha tocoferol.

### Other Components

Other components may include diluents such as cellulose, microcrystalline cellulose, hydroxypropyl cellulose, starch, hydroxypropylmethyl cellulose and the like. Excipients can be added to adjust the tonicity of the composition, such as sodium chloride, glucose, dextrose, mannitol, sorbitol, lactose and the like. Acidic or basic buffers can also be added to control the pH. Co-solvents or solubilizers such as glycerol, polyethylene glycols, polyethylene glycols derivatives, polyethylene glycol 660 hydroxystearate (Solutol HS15 from BASF), butylene glycol, hexylene glycol, and the like, can also be added.

### Transdermal Compositions

The compositions for transdermal administration include a compound of the invention including fatty acid salts, and optionally can also include other ingredients including, but not limited to, carriers and excipients, such as permeation enhancers which promote transdermal absorption of the active ingredient after transdermal administration.

The amount of active ingredient absorbed depends on many factors. These factors include the active ingredient concentration, the active ingredient delivery vehicle, the skin contact time, the area of the skin dosed, the ratio of the ionized and unionized forms of the active ingredient at the pH of the absorption site, the molecular size of the active ingredient molecule, and the active ingredient's relative lipid solubility.

### Transdermal Devices

The transdermal device for delivering the active ingredients described herein can be of any type known in the art, including the monolithic, matrix, membrane, and other types typically useful for administering active ingredients by the transdermal route. Such devices are disclosed in U.S. Pat. Nos. 3,996,934; 3,797,494; 3,742,951; 3,598,122; 3,598,123; 3,731,683; 3,734,097; 4,336,243; 4,379,454; 4,460,372; 4,486,193; 4,666,441; 4,615,699; 4,681,584; and 4,558,580 among others.

These devices tend to be flexible, adhere well to the skin, and have a polymeric backing (covering) that is impermeable to the active ingredient to be delivered, so that the active ingredient is administered uni-directionally through the skin. The active ingredient, or pharmaceutically acceptable salt thereof, is typically present in a solution or dispersion, which can be in the form of a gel, a solution, or a semi-solid, and which aids in active ingredient delivery through the stratum corneum of the epidermis and to the dermis for absorption.

### Membrane Devices

Membrane devices typically have four layers: (1) an impermeable backing, (2) a reservoir layer, (3) a membrane layer (which can be a dense polymer membrane or a microporous membrane), and (4) a contact adhesive layer which either covers the entire device surface in a continuous or discontinuous coating or surrounds the membrane layer. Examples of materials that may be used to act as an impermeable layer are high, medium, and low density polyethylene, polypropylene, polyvinylchloride, polyvinylidene chloride, polycarbonate, polyethylene terepthalate, and polymers laminated or coated with aluminum foil. Others are disclosed in the standard transdermal device patents mentioned herein. In certain embodiments in which the reservoir layer is fluid or is a polymer, the outer edge of the backing layer can overlay the edge of the reservoir layer and be sealed by adhesion or fusion to the diffusion membrane layer. In such instances, the reservoir layer need not have exposed surfaces.

The reservoir layer is underneath the impermeable backing and contains a carrier liquid, typically water and/or an alcohol, or polyol or ester thereof, and may or may not contain the active ingredients. The reservoir layer can include diluents, stabilizers, vehicles, gelling agents, and the like in addition to the carrier liquid and active ingredients.

The diffusion membrane layer of the laminate device can be made of a dense or microporous polymer film that has the requisite permeability to the active ingredient and the carrier liquid. Preferably, the membrane is impermeable to ingredients other than the active ingredient and the carrier liquid, although when buffering at the skin surface is desired, the membrane should be permeable to the buffer in the composition as well. Examples of polymer film that may be used to make the membrane layer are disclosed in U.S. Pat. Nos. 3,797,454 and 4,031,894. The preferred materials are polyurethane, ethylene vinyl alcohol polymers, and ethylene/vinyl acetate.

### Monolithic Matrices

The second class of transdermal systems is represented by monolithic matrices. Examples of such monolithic devices are U.S. Pat. Nos. 4,291,014; 4,297,995; 4,390,520 and 4,340,043. Others are known to those of ordinary skill in this art.

Monolithic and matrix type barrier transdermal devices typically include: (1) Porous polymers or open-cell foam polymers, such as polyvinyl chloride (PVC), polyurethanes, polypropylenes, and the like; (2) Highly swollen or plasticized polymers such as cellulose, HEMA or MEMA or their copolymers, hydroxypropyl methylcellulose (HPMC), hydroxyethyl methylcellulose (HEMC), and the like, polyvinyl alcohol (PVA)/polyvinylpyrollidone (PVP), or other hydrogels, or PVC, polyurethane, ethylene/vinyl acetate, or their copolymers; (3) Gels of liquids, typically including water and/or hydroxyl-containing solvents such as ethanol, and often containing gelling agents such PVP, carboxymethylcellulose (CMC), hydroxypropylcellulose such as sold under the tradename Klucel®, HPMC, alginates, kaolinate, bentonite, or montmorillonite, other clay fillers, stearates, silicon dioxide particles, and the like; (4) Nonwoven materials made of textiles, celluloses, polyurethanes, polyester, or other fiber; (5) Sponges, which can be formed from natural or foamed polymers; and (6) Adhesives, ideally dermatologically-acceptable pressure sensitive adhesives, for example, silicone adhesives or acrylic adhesives.

### Polymeric Barrier Materials

Representative polymeric barrier materials include, but are not limited to: Polycarbonates, such as those formed by phosgenation of a dihydroxy aromatic such as bisphenol A, including materials are sold under the trade designation Lexan® (the General Electric Company); Polyvinylchlorides, such as Geon® 121 (B. G. Goodrich Chemical Company); Polyamides (“nylons”), such as polyhexamethylene adipamide, including NOMEX® (E. I. DuPont de Nemours & Co.).

Modacrylic copolymers, such as DYNEL®, are formed of polyvinylchloride (60 percent) and acrylonitrile (40 percent), styrene-acrylic acid copolymers, and the like. Polysulfones, for example, those containing diphenylene sulfone groups, for example, P-1700 (Union Carbide Corporation). Halogenated polymers, for example, polyvinylidene fluoride, such as Kynar® (Pennsalt Chemical Corporation), polyvinylfluoride, such as Tedlar® (E. I. DuPont de Nemours & Co.), and polyfluorohalocarbons, such as Aclar® (Allied Chemical Corporation). Polychlorethers, for example, Penton® (Hercules Incorporated), and other thermoplastic polyethers. Acetal polymers, for example, polyformaldehydes, such as Delrin® (E. I. DuPont de Nemours & Co.). Acrylic resins, for example, polyacrylonitrile, polymethyl methacrylate (PMMA), poly n-butyl methacrylate, and the like.

Other polymers such as polyurethanes, polyimides, polybenzimidazoles, polyvinyl acetate, aromatic and aliphatic, polyethers, cellulose esters, e.g., cellulose triacetate; cellulose; colledion (cellulose nitrate with 11% nitrogen); epoxy resins; olefins, e.g., polyethylene, polypropylene; polyvinylidene chloride; porous rubber; cross linked poly(ethylene oxide); cross-linked polyvinylpyrrolidone; cross-linked polyvinyl alcohol); polyelectrolyte structures formed of two ionically associated polymers of the type as set forth in U.S. Pat. Nos. 3,549,016 and 3,546,141; derivatives of polystyrene such as poly(sodium styrenesulfonate) and poly(vinylbenzyltrimethyl-ammonium chloride); poly(hydroxyethylmethacrylate); poly(isobutylvinyl ether), and the like, may also be used. A large number of copolymers which can be formed by reacting various proportions of monomers from the above list of polymers are also useful. If the membrane or other barrier does not have a sufficiently high flux, the thickness of the membrane or barrier can be reduced. However, the thickness should not be reduced to the point where it is likely to tear, or to a point where the amount of active ingredient which can be administered is too low.

### Adhesives

The transdermal drug delivery compositions typically include a contact adhesive layer to adhere the device to the skin. The active agent may, in some embodiments, reside in the adhesive. Adhesives include polyurethanes; acrylic or methacrylic resins such as polymers of esters of acrylic or methacrylic acid with alcohols such as n-butanol, n-pentanol, isopentanol, 2-methylbutanol, 1-methylbutanol, 1-methylpentanol, 2-methylpentanol, 3-methylpentanol, 2-ethylbutanol, isooctanol, n-decanol, or n-dodecanol, alone or copolymerized with ethylenically unsaturated monomers such as acrylic acid, methacrylic acid, acrylamide, methacrylamide, N-alkoxymethyl acrylamides, N-alkoxymethyl methacrylamides, N-tertbutylacrylamide, itaconic acid, vinylacetate, N-branched alkyl maleamic acids wherein the alkyl group has 10 to 24 carbon atoms, glycol diacrylates, or mixtures of these; natural or synthetic rubbers such as styrenebutadiene, butylether, neoprene, polyisobutylene, polybutadiene, and polyisoprene; polyvinylacetate; unreaformaldehyde resins; phenolformaldehyde resins; resorcinol formaldehyde resins, cellulose derivatives such as ethylcellulose, methylcellulose, nitrocellulose, cellulose acetatebutyrate, and carboxymethyl cellulose; and natural gums such as guar, acacia, pectins, starch, dextrin, albumin, gelatin, casein, etc. The adhesives can be compounded with tackifiers and stabilizers, as is well known in the art.

Representative silicone adhesives include silicone elastomers based on monomers of silanes, halosilanes, or CMS alkoxysilanes, especially polydimethylsiloxanes which may be used alone or formulated with a silicone tackifier or silicone plasticizer which are selected from medically acceptable silicone fluids, i.e. non-elastomeric silicones based on silanes, halosilanes or C₁₋₁₈ alkoxysilanes. Typical silicone adhesives are available from Dow Corning under the tradename SILASTIC®.

### Liquid Vehicles

Transdermal compositions can include a variety of components, including a liquid vehicle, typically a C₂₋₄ alkanol such as ethanol, isopropanol, n-propanol, butanol, a polyalcohol or glycol such as propylene glycol, butylene glycol, hexylene glycol, ethylene glycol, and/or purified water. The vehicle is typically present in an amount of between about 5 and about 75% w/w, more typically, between about 15.0% and about 65.0% w/w, and, preferably, between about 20.0 and 55.0% w/w.

Water augments the solubility of hydrophilic active agents in the composition, and accelerates the release of lipophilic active agents from a composition. Alcohols, such as ethanol, increase the stratum corneum liquid fluidity or function to extract lipids from the stratum corneum. As discussed herein, the glycols can also act as permeation enhancers.

### Controlled Release of the Active Agent

The administration of the active agent can be controlled by using controlled release compositions, which can provide rapid or sustained release, or both, depending on the compositions. There are numerous particulate drug delivery vehicles known to those of skill in the art which can include the active ingredients, and deliver them in a controlled manner. Examples include particulate polymeric drug delivery vehicles, for example, biodegradable polymers, and particles formed of non-polymeric components. These particulate drug delivery vehicles can be in the form of powders, microparticles, nanoparticles, microcapsules, liposomes, and the like. Typically, if the active agent is in particulate form without added components, its release rate depends on the release of the active agent itself. In contrast, if the active agent is in particulate form as a blend of the active agent and a polymer, the release of the active agent is controlled, at least in part, by the removal of the polymer, typically by dissolution or biodegradation.

In one embodiment, the transdermal compositions can provide an initial rapid release of the active ingredient followed by a sustained release of the active ingredient. U.S. Pat. No. 5,629,011 provides examples of this type of composition. There are numerous transdermal compositions that use transdermal delivery to deliver nicotine in a time-release manner (such as rate-controlling membranes), including currently marketed nicotine replacement therapies. These are also suitable for administering the compounds described herein.

### Semi-Solid Dosage Forms

In one embodiment, the transdermal dosage form is not a “patch,” but rather, a semisolid dosage form such as a gel, cream, ointment, liquid, etc. In this embodiment, one can augment patient's compliance and cover a broader surface area than can be covered with a patch.

In this embodiment, particularly when used for pain treatment, the dosage form can include other active and inactive components typically seen in semisolid dosage forms used to treat pain. These include, but are not limited to, menthol, wintergreen, capsaicin, aspirin, NSAIDs, narcotic agents (e.g. fentanyl), alcohols, oils such as emulsion oil, and solvents such as DMSO.

### Iontophoresis

In addition to delivery via transdermal drug delivery devices and semi-solid dosage forms, the active ingredients can also be delivered via iontophoresis. Iontophoresis is a non-invasive method of propelling high concentrations of a charged substance, such as the active ingredients described herein, transdermal̂ by repulsive electromotive force. The technique involves using a small electrical charge applied to an iontophoretic chamber containing a similarly charged active agent and its vehicle. The skin's permeability is altered upon application of the charge, and this increases migration of the active ingredient into the epidermis.

Iontophoresis can be used to transdermally deliver the active agents, using active transportation within an electric field, typically by electromigration and electroosmosis. These movements are typically measured in units of chemical flux, commonly μmol/cm²*h. The isoelectric point of the skin is approximately 4. Under physiological conditions, where the surface of the skin is buffered at or near 7.4, the membrane has a net negative charge, and electroosmotic flow is from anode (−) to cathode (+). Electroosmosis augments the anodic delivery of the (positively charged) active agents described herein.

Iontophoresis devices include two electrodes, which are typically attached to a patient, each connected via a wire to a microprocessor controlled electrical instrument. The active agents are placed under one or both of the electrodes, and are delivered into the body as the instrument is activated.

Typically, ions are delivered into the body from an aqueous drug reservoir contained in the iontophoretic device, and counter ions of opposite charge are delivered from a “counter reservoir.” Solutions containing the active ingredient, and also solutions of the counter ions, can be stored remotely and introduced to an absorbent layer of the iontophoresis electrode at the time of use. Examples of such systems are described in U.S. Pat. Nos. 5,087,241; 5,087,242; 5,846,217; and 6,421,561, the contents of which are hereby incorporated by reference. Alternatively, as described in U.S. Pat. No. 5,685,837, the active agents can be pre-packaged in dry form into the electrode(s). This approach requires a moisture activation step at the time of use.

Solutions of the active agents can be co-packaged with the iontophoretic device, ideally positioned apart from the electrodes and other metallic components until the time of use. This technique, and suitable devices, are described, for example, in U.S. Pat. Nos. 5,158,537; 5,288,289; 5,310,404; 5,320,598; 5,385,543; 5,645,527; 5,730,716; and 6,223,075. In these devices, a co-packaged electrolyte constituent liquid is stored remotely from the electrodes, in a rupturable container and a mechanical action step at the time of use induces a fluid transfer to a receiving reservoir adjacent to the electrodes. These systems enable precise fluid volumes to be incorporated at the time of manufacture to avoid overfilling.

In addition to solutions, the active agents can be present in a pre-formed gel, as described in U.S. Pat. No. 4,383,529, incorporated by reference. Thus, a preformed gel containing the active agent can be transferred into an electrode receptacle at the time of use. This system can be advantageous in that it provides a precise pre-determined volume of the gel, thus preventing over-filling. Further, since the active agent is present in a gel composition, it is less likely to leak during storage or transfer.

In some embodiments, the transdermal drug delivery is carried out using devices that include a polymeric barrier, adhered to the skin with a suitable adhesive, and which also include a suitable amount of the active ingredients, or salts thereof, in solution or dispersion and in contact with the skin or a rate-controlling membrane may be used between the active-containing composition and the skin. In others, the delivery is carried out using semisolid compositions, such as cremes or lotions, which include the active ingredients, and which are applied to the skin. In still other embodiments, the active ingredients are delivered using iontophoresis, wherein the positively charged active agents are administered by electroosmosis. There may also be embodiments wherein the active ingredient(s) is formulated within the matrix of the adhesive.

As previously indicated, the present invention provide transdermal compositions of (4aR,10aR)-1-n-propyl-1,2,3,4,4a,5,10,10a-octahydro-benzo[g]quinoline-6,7-diol and related compounds, which may be delivered to the systemic circulation via delivery across the skin.

In one embodiment, the composition is further characterized as patch. In one embodiment, the composition is further characterized as a semisolid dosage form. In one embodiment, the composition is further characterized as a gel, lotion or creme. In one embodiment, the composition is further characterized as a controlled release formulation. In one embodiment, the composition is further comprising a permeation enhancer. In one embodiment, the composition is further comprising one or more adhesive, binder, lubricant, glidant, disintegrant or mixture thereof.

The compound of the invention as a pharmaceutical composition for transdermal administration may be administered in any suitable way across the skin, and the compound may be presented in any suitable dosage form for such administration, e.g. in form of simple solutions or dispersions, simple tablets, matrix tablets, capsules, powders, syrups, dissolvable films, patches, lipophilic gels. In another embodiment, the compound of the invention is administered in the form of a dissolvable film.

In a specific embodiment of the invention, there is provided a transdermal composition comprising a therapeutically effective amount of the compound of the invention, or a pharmaceutically acceptable acid addition salt thereof, for administration across the skin.

In a further aspect the invention provides the use of said composition for the preparation of a medicament for the treatment of neurodegenerative disorders such as Parkinson's disease and Huntington's disease.

In a further aspect the invention provides the use of the transdermal composition for the preparation of a medicament for the treatment of psychoses, impotence, renal failure, heart failure or hypertension.

In another aspect the invention provides the use of the transdermal composition for the manufacture of a medicament for the treatment of cognitive impairment in a mammal.

In a still further aspect the invention provides the use of the transdermal composition for the manufacture of a medicament for the treatment of restless legs syndrome (RLS) or periodic limb movement disorder (PLMD).

In a still further aspect the invention provides the use of the transdermal composition for the manufacture of a medicament for the treatment of erectile dysfunction.

In a different aspect the invention provides the use of the transdermal composition for the manufacture of a medicament for the treatment of movement disorders, poverty of movement, dyskinetic disorders, gait disorders or intention tremor in a mammal.

In a further aspect the invention provides the use of the transdermal composition for the treatment of neurodegenerative disorders such as Parkinson's disease and Huntington's disease.

In a further aspect the invention provides the use of the transdermal composition for the treatment of psychoses, impotence, renal failure, heart failure or hypertension.

In another aspect the invention provides the use of the transdermal composition for the treatment of cognitive impairment in a mammal.

In a still further aspect the invention provides the use of the transdermal composition for the treatment of restless legs syndrome (RLS) or periodic limb movement disorder (PLMD).

In a different aspect the invention provides the use of the transdermal composition for the treatment of movement disorders, poverty of movement, dyskinetic disorders, gait disorders or intention tremor in a mammal.

In separate aspects the invention provides the use of the transdermal composition for the manufacture of medicaments, which are intended for administration via the skin.

The invention also provides a method of treating a mammal suffering from a neurodegenerative disorder such as Parkinson's disease and Huntington's disease comprising administering to the mammal a therapeutically effective amount of the transdermal composition.

In another aspect the invention also provides a method of treating a mammal suffering from psychoses, impotence, renal failure, heart failure or hypertension, comprising administering to the mammal a therapeutically effective amount of the transdermal composition.

In a further aspect the invention provides a method of treating a mammal suffering from a cognitive impairment, comprising administering to the mammal an effective amount of the transdermal composition.

The invention also relates to a method of treating a mammal suffering from restless legs syndrome (RLS) or periodic limb movement disorder (PLMD), comprising administering to the mammal a transdermal composition of the compound of the invention, or a pharmaceutically acceptable addition salt thereof.

The invention also relates in a separate aspect to a method of treating a mammal suffering from movement disorders, poverty of movement, dyskinetic disorders, gait disorders or intention tremor comprising administering to the mammal of the pharmaceutical composition.

The therapeutically effective amount of the compound of the invention, calculated as the daily dose of the compound of the invention above as the free base, is suitably between 0.001 and 12.5 mg/day, more suitable between 0.005 and 10.0 mg/day, e.g. preferably between 0.01 and 5.0 mg/day. In a specific embodiment the daily dose of the compound of the invention is between 0.1 and 1.0 mg/day.

In another embodiment the daily dose of the compound of the invention is less than about 0.1 mg/day. In a separate embodiment the daily dose of the compound of the invention is about 0.01 mg/day. In a further embodiment the invention provides a formulation comprising from 0.0001 mg to 12.5 mg of the compound of the invention for transdermal delivery. In a further embodiment the invention provides a formulation comprising from 0.0001 mg to 0.01 mg of the compound of the invention for transdermal delivery. In a further embodiment the invention provides a formulation comprising from 0.001 mg to 0.10 mg of the compound of the invention for transdermal delivery. In a further embodiment the invention provides a formulation comprising from 0.01 mg to 1.0 mg of the compound of the invention for transdermal delivery.

Ultimately, the exact dose of the compound of the invention and the particular formulation to be administered depend on a number of factors, e.g., the condition to be treated, the desired duration of the treatment and the rate of release of the active agent. For example, the amount of the active agent required and the release rate thereof may be determined on the basis of known in vitro or in vivo techniques, determining how long a particular active agent concentration in the blood plasma remains at an acceptable level for a therapeutic effect.

### Pharmaceutically Acceptable Salts of Compound 10

Compound 10 and related compounds form pharmaceutically acceptable acid addition salts with a wide variety of organic and inorganic acids. Such salts are also part of this invention. A pharmaceutically acceptable acid addition salt of the compound of the invention is formed from a pharmaceutically acceptable acid as is well known in the art. Such salts include the pharmaceutically acceptable salts listed in Journal of Pharmaceutical Science, 66, 2-19 (1977) and are known to the skilled person. Typical inorganic acids used to form such salts include hydrochloric, hydrobromic, hydroiodic, nitric, sulphuric, phosphoric, hypophosphoric, metaphosphoric, pyrophosphoric, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include the chloride, bromide, iodide, nitrate, acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, isobutyrate, phenylbutyrate, hydroxybutyrate, butyne-1,4-dicarboxylate, hexyne-1,4-dicarboxylate, caprate, caprylate, cinnamate, citrate, formate, fumarate, glycollate, heptanoate, hippurate, lactate, malate, maleate, hydroxymaleate, malonate, mandelate, mesylate, nicotinate, isonicotinate, oxalate, phthalate, teraphthalate, propiolate, propionate, phenylpropionate, salicylate, sebacate, succinate, suberate, benzenesulfonate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, p-toluenesulfonate, xylenesulfonate, tartrate, and the like.

### BRIEF DESCRIPTION OF THE FIGURES

FIG. 1: Crystal structure of compound ent-10. The absolute configuration was determined by the anomalous scattering of the ‘heavy’ bromine atom.

FIG. 2: Dose-response curve for the concentration-dependent stimulation of intracellular Ca²⁺ release by dopamine in hD₅-transfected CHO-Ga16 cells.

FIG. 3: Representative Chromatogram of Sample from animal 2, Day 5

FIG. 4: Dose Normalised AUC0-∞ for Compound 10 from Example 14

FIG. 5: Dose Normalised Cmax for Compound 10 from Example 14

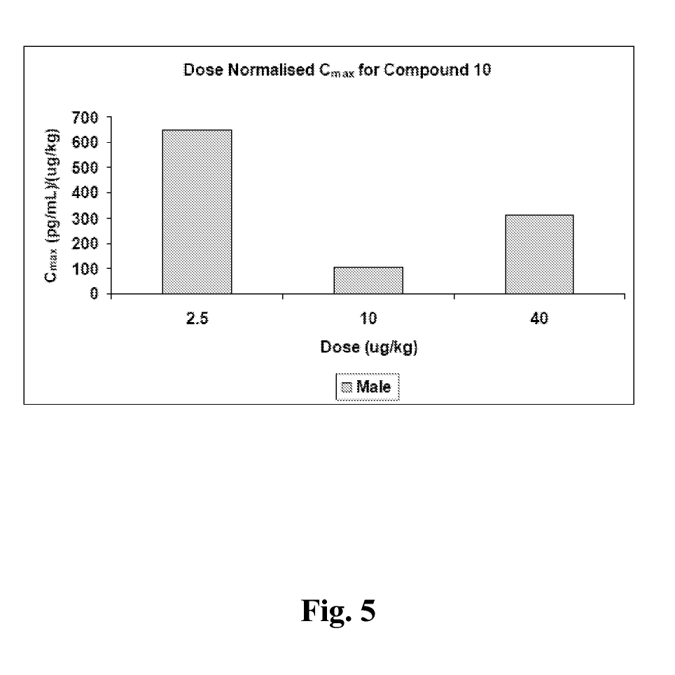

### EXPERIMENTAL SECTION

Analytical LC/MS data were obtained on a PE Sciex API 150EX instrument equipped with atmospheric pressure photo ionization and a Shimadzu LC-8A/SLC-10A LC system. Purity was determined by integration of the UV (254 nm) and ELSD traces. MS instruments are from Peskier (API), equipped with APPI-source and operated in positive ion mode. The retention times in the UV-trace (RT) are expressed in min. Solvents A was made of 0.05% TFA in water, while solvent B was made of 0.035% TFA and 5% water in acetonitrile. Several different methods have been used:

Method 25: API 150EX and Shimadzu LC10AD/SLC-10A LC system. Column: dC-18 4.6×30 mm, 3 microm (Atlantis, Waters). Column temperature: 40° C. Gradient: reverse phase with ion pairing. Flow: 3.3 mL/min. Injection volume: 15 microL. Gradient: 2% B in A to 100% B over 2.4 min then 2% B in A for 0.4 min. Total run time: 2.8 min.

Method 14: API 150EX and Shimadzu LC8/SLC-10A LC system. Column: C-18 4.6×30 mm, 3.5 microm (Symmetry, Waters). Column temperature: rt. Gradient: reverse phase with ion pairing. Flow: 2 mL/min. Injection volume: 10 microL. Gradient: 10% B in A to 100% B over 4 min then 10% B in A for 1 min. Total run time: 5 min.

X-ray crystal structure determination was performed as follows. The crystal of the compound was cooled to 120 K using a Cryostream nitrogen gas cooler system. The data were collected on a Siemens SMART Platform diffractometer with a CCD area sensitive detector. The structures were solved by direct methods and refined by full-matrix least-squares against F² of all data. The hydrogen atoms in the structures could be found in the electron density difference maps. The non-hydrogen atoms were refined anisotropically. All the hydrogen atoms were at calculated positions using a riding model with O—H=0.84, C—H=0.99-1.00, N—H=0.92-0.93 Å. For all hydrogen atoms the thermal parameters were fixed [U(H)=1.2 U for attached atom]. The Flack x-parameters are in the range 0.0(1)-0.05(1), indicating that the absolute structures are correct. Programs used for data collection, data reduction and absorption were SMART, SAINT and SADABS [cf. “SMART and SAINT, Area Detector Control and Integration Software”, Version 5.054, Bruker Analytical X-Ray Instruments Inc., Madison, USA (1998), Sheldrick “SADABS, Program for Empirical Correction of Area Detector Data” Version 2.03, University of Göttingen, Germany (2001)]. The program SHELXTL [cf. Sheldrick “SHELXTL, Structure Determination Programs”, Version 6.12, Bruker Analytical X-Ray Instruments Inc., Madison, USA (2001)] was used to solve the structures and for molecular graphics.

### Synthesis of the Compounds of the Invention

Starting from compound 1 whose synthesis is described in the literature prepared as described in Taber et al., J. Am. Chem. Soc., 124 (42), 12416 (2002), compound 8 can be prepared as described herein in eight steps. This material can be resolved by chiral SFC as described herein to give compounds 9 and ent-9. After cleavage of the Boc-protective group, reductive amination can be used to introduce the n-propyl group on the nitrogen atom. The resulting masked catechol amines can be deprotected under standard conditions by treatment with 48% HBr or by reaction with BBr₃ to give compounds 10 and ent-10.

The enantiomer of example 1 (compound 10) and ent-example 1 (ent-compound 10), can be prepared in a similar manner from ent-9. The racemate of example 1, rac-example 1, can be prepared by mixing a 1:1 mixture of example 1 and ent-example 1. It can also be obtained from non-resolved compound 8 or a 1:1 mixture of compound 9/ent-9 as described above for the pure enantiomers. Alternatively, rac-example 1 can be prepared as described in the literature (Cannon et al., J. Heterocycl. Chem. 17, 1633 (1980)).

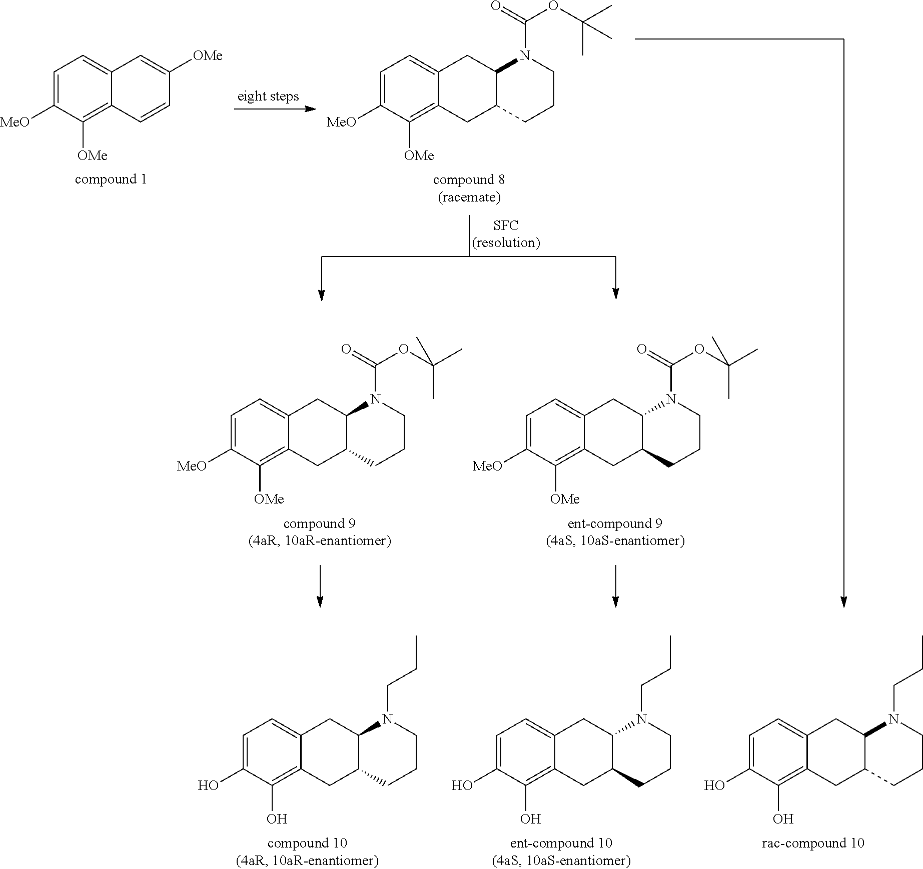

Synthesis of compounds 9 and ent-9.

### 7-Iodo-1,2,6-trimethoxy-naphthalene (compound 2)

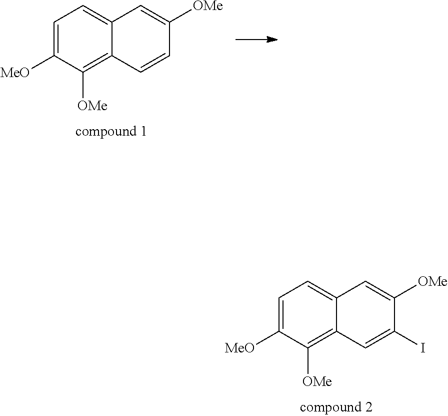

To a stirred solution of compound 1 (26.2 g; prepared as described in Taber et al., J. Am. Chem. Soc., 124 (42), 12416 (2002)) in dry THF (200 mL) under argon and at −78° C. was slowly added s-butyl lithium (1.2 M in cyclohexane, 110 mL). The solution was stirred at −78° C. for 3 h. A solution of iodine (30.5 g) in dry THF (50 mL) was added over a period of 10 min. The resulting mixture was then stirred for another 10 min at −78° C. The reaction mixture was quenched by the addition of sat. NH₄Cl (100 mL), water (240 mL), and Et₂O (240 mL). The organic layer was washed with 10% aqueous sodium sulfite solution (100 mL), dried (Na₂SO₄) and concentrated in vacuo. The crude material was purified by distilling off unreacted starting material. The residue was further purified by silica gel chromatography (EtOAc/heptane) to produce an impure solid material, which was purified by precipitation from EtOAc/heptane affording 11.46 g of compound 2.

### (E/Z)-3-(3,7,8-Trimethoxy-naphthalen-2-yl)-acrylonitrile (compound 3)

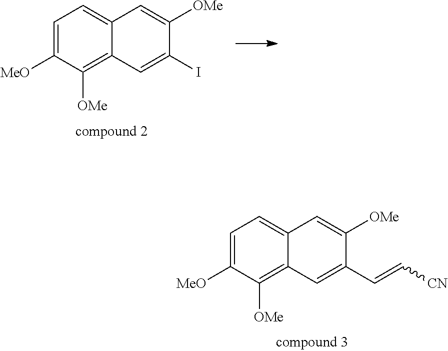

To a suspension of compound 2 (3.41 g) in dry acetonitrile (10.7 mL) in a microwave reactor vial was added acrylonitrile (1.19 mL) Pd(OAc)₂ (73 mg), and triethylamine (1.48 mL). The vial was sealed, and the mixture was heated for 40 min at 145° C. under microwave irradiation. This procedure was carried out two more times (using a total of 10.23 g of compound 5). The crude reaction mixtures were combined and the catalyst was filtered off, and the filtrate was concentrated in vacuo. The residue was partitioned between Et₂O (300 mL) and 2M HCl (150 mL). The organic layer was washed with brine (100 mL), dried (Na₂SO₄) and concentrated in vacuo. The crude material (7.34 g) was purified by silica gel chromatography (EtOAc/heptane) to produce 5.23 g of compound 3 as a mixture of olefin isomers.

### 3-(3,7,8-Trimethoxy-naphthalen-2-yl)-propionitrile (compound 4)

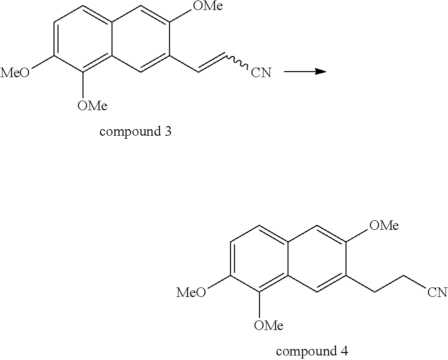

Compound 3 (5.23 g) was dissolved in CHCl₃ (15 mL) and 99% EtOH (100 mL). 10% Pd/C (0.8 g) was added and the solution was hydrogenated for 45 min under a hydrogen pressure of 3 bar using a Parr shaker. The catalyst was filtered off, and the filtrate was passed through a small plough of silica gel (eluent: 99% EtOH). Yield: 4.91 g compound 4 as a white solid.

### [3-(3,7,8-Trimethoxy-1,4-dihydro-naphthalen-2-yl)-propyl]-carbamic acid t-butyl ester (compound 5)

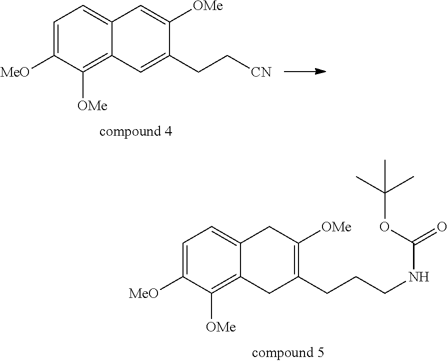

Compound 4 (5.0 g) was dissolved in 99% EtOH (150 mL) and the mixture was heated to reflux under nitrogen atmosphere. Sodium metal (5 g) was added in small lumps over 3 h. The mixture was refluxed for an additional 2 h, before it was stirred at rt for 2 days. Then it was heated to reflux again, and more sodium metal (3.68 g) was added and the mixture was refluxed overnight. After cooling on an ice/water bath, the reaction was quenched by the addition of solid ammonium chloride (20 g) and water (25 mL). The resulting mixture was filtered, and the filtrate was concentrated in vacuo. The residue was partitioned between diethyl ether (50 mL) and water (50 mL). The aqueous layer was neutralized with 37% HCl and extracted with diethyl ether (2×50 mL). The combined organic extracts were washed with brine (50 mL), dried (MgSO₄) and concentrated in vacuo to afford an oil. This material was dissolved in THF (50 mL) and treated with Boc₂O (2.34 g) and Et₃N (1.78 mL) at rt. After six days the volatiles were removed in vacuo and the residue was purified by silica gel chromatography (EtOAc/heptane). This provided impure compound 5 (1.52 g).

### Racemic 6,7-dimethoxy-2,3,4,4a,5,10-hexahydro-benzo[g]quinoline hydrochloride (compound 6)

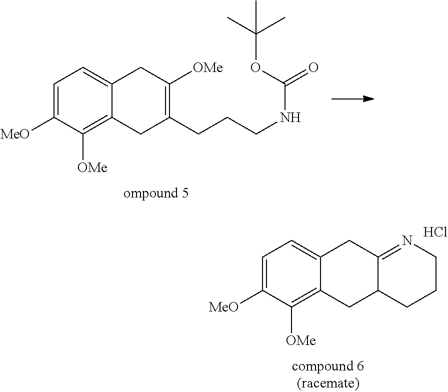

Compound 5 (1.52 g from the previous step) was dissolved in MeOH (20 mL). 37% HCl (3.5 mL) was added, and the mixture was refluxed for 4 h. The volatiles were removed in vacuo, using toluene to azeotropically remove the water. This provided impure compound 6 (0.89 g) as an yellow oil.

### Racemic trans-6,7-dimethoxy-3,4,4a,5,10,10a-hexahydro-2H-benzo[g]quinoline-1-carboxylic acid t-butyl ester (compound 8)

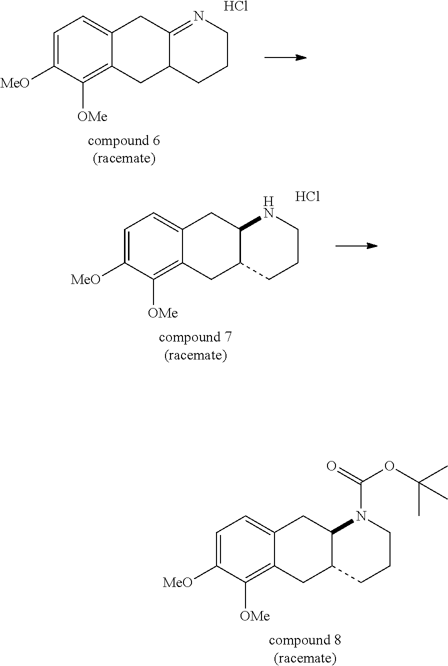

Compound 6 (0.89 g) was dissolved in MeOH (10 mL) and NaCNBH₃ (0.19 g) was added. The reaction was stirred overnight at rt. The crude mixture was cooled on an ice/water bath, before it was quenched with 2 M HCl in Et₂O (1 mL). The mixture was partitioned between Et₂O (50 mL), water (50 mL), and 2 M NaOH (10 mL). The aqueous layer was extracted with diethyl ether (3×50 mL). The combined organic layers were dried (MgSO₄) and concentrated in vacuo to afford the impure free amine (compound 7). This material was dissolved in THF (25 mL) and treated with Boc₂O (0.68 g) and Et₃N (0.86 mL) at rt for 1 h. The crude mixture was concentrated in vacuo, and the residue was purified by silica gel chromatography (EtOAc/heptane) to provide 1.18 g of racemic compound 8 sufficiently pure for the next step.

### SFC-separation of the enantiomers of racemic trans-6,7-dimethoxy-3,4,4a,5,10,10a-hexahydro-2H-benzo[g]quinoline-1-carboxylic acid t-butyl ester (compounds 9 and ent-9)

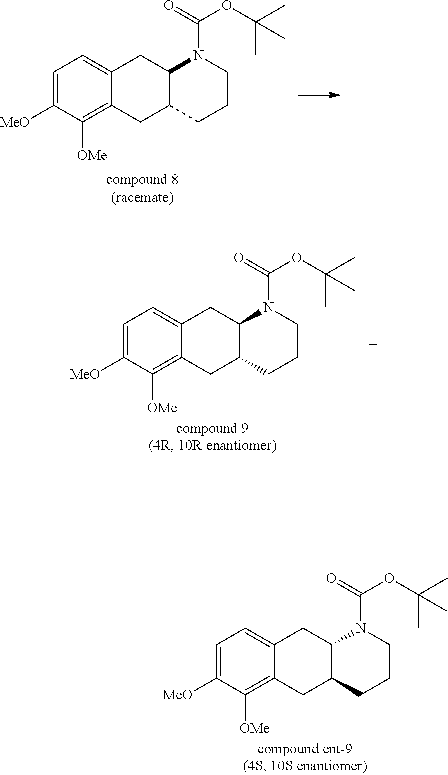

Compound 8 (19.7 g) was resolved into its enantiomers using chiral SFC on a Berger SFC multigram II instrument equipped with a Chiralcel OD 21.2×250 mm column. Solvent system: CO₂/EtOH (85:15), Method: constant gradient with a flow rate of 50 mL/min. Fraction collection was performed by UV 230 nm detection. Fast eluting enantiomer (4aR,10aR enantiomer; compound 9): 9.0 g of a white solid. Slow eluting enantiomer (4aS,10aS enantiomer; compound ent-9): 8.1 g of a white solid.

### (4aS,10aS)-6,7-Dimethoxy-1,2,3,4,4a,5,10,10a-octahydro-benzo[g]quinoline hydrochloride (compound ent-9′)

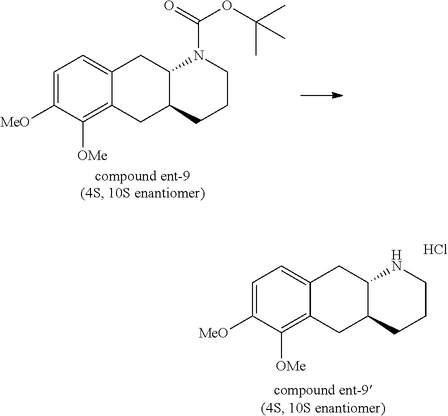

Compound ent-9 (0.52 g) was dissolved in MeOH (15 mL) and treated with 5 M HCl in Et₂O (7.5 mL) at rt for 2 h. The mixture was concentrated in vacuo and the solid was dried in vacuo to give compound ent-9′ as a white solid. LC/MS (method 14): RT 1.31 min.

### Example 1

### Preparation of the Compounds of the Invention

### Synthesis of (4aR,10aR)-1-n-Propyl-1,2,3,4,4a,5,10,10a-octahydro-benzo[g]quinoline-6,7-diol hydrobromide (compound 10)

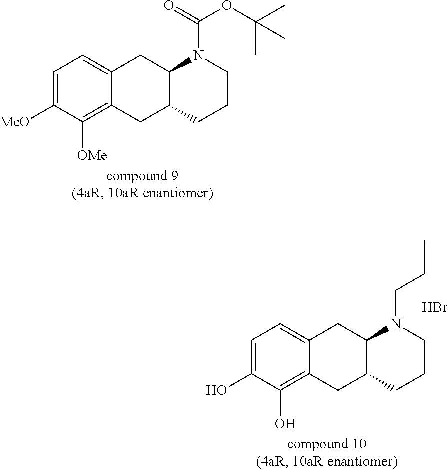

Compound 9 (0.5 g) was dissolved in 99% EtOH (5 mL) and treated with 2M HCl in Et₂O (4 mL) overnight at rt. The crude mixture was concentrated in vacuo, and the residue was partitioned between EtOAc and 10% aqueous NaOH (5 mL). The aqueous layer was extracted with EtOAc, and the combined organic layers were washed with brine, dried (MgSO₄), concentrated in vacuo. The residue was dissolved in 99% EtOH (5 mL) and treated with propionic aldehyde (0.52 mL), NaCNBH₃ (0.45 g), and AcOH (3 drops) overnight at rt. The crude mixture was portioned between sat. aqueous NaHCO₃ (12.5 mL), water (12.5 mL), and EtOAc (2×25 mL). The combined organic layers were washed with brine, dried (MgSO₄), and concentrated in vacuo. The residue was purified by silica gel chromatography (MeOH/EtOAc). The obtained intermediate was treated with 48% HBr (3 mL) at 150° C. for 1 h under microwave conditions, before the crude mixture was stored at 4° C. overnight. The precipitated material was isolated by filtration and dried in vacuo. Yield of compound 10: 103 mg as a solid. LC/MS (method 25): RT 0.77 min.

### (4aS,10aS)-1-Propyl-1,2,3,4,4a,5,10,10a-octahydro-benzo[g]quinoline-6,7-diol hydrobromide (compound ent-10)

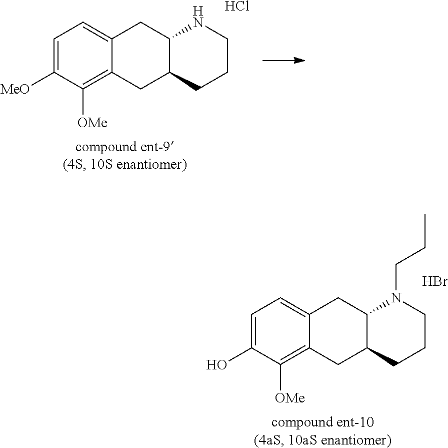

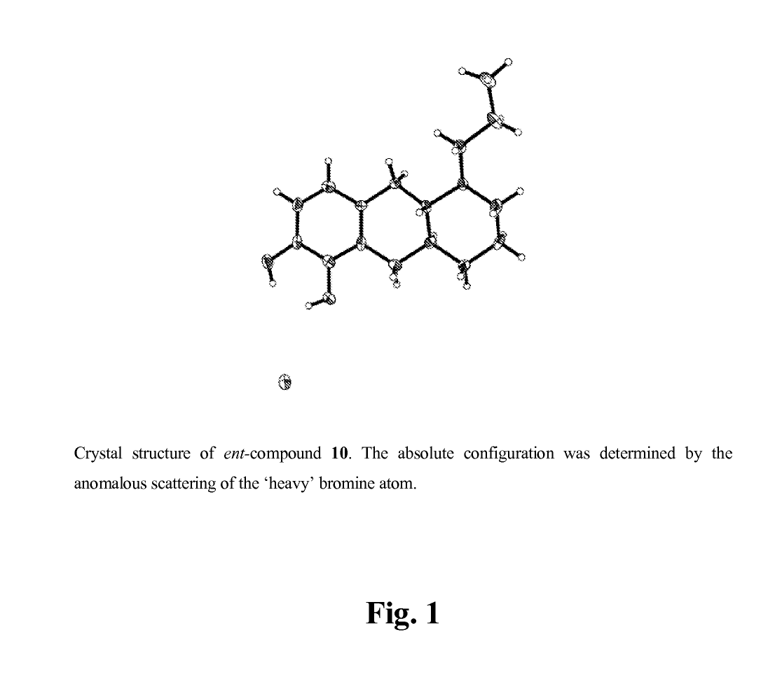

The procedure described for compound 10 was followed starting from compound ent-9′ (0.5 g; the HCl salt was liberated by partitioning between EtOAc and 10% aqueous NaOH before the reductive amination step). Yield of compound ent-10: 70 mg as a solid. LC/MS (method 25): RT 0.70 min. A small sample of compound ent-10 was dissolved in MeOH and allowed to crystallize slowly at rt over 2 months. The formed white crystals were collected and subjected to X-ray analysis (cf. FIG. 1). The absolute configuration of compound ent-10 was determined by X-ray crystallography and allowed for unambiguous determination of the stereochemistry of compounds 9 and 10 and hence their related compounds.

### Example 2

### General Diester syntheses

The scheme below provides a general procedure for the conversion of catecholamines to the symmetric, asymmetric and mono esters of compound 10.

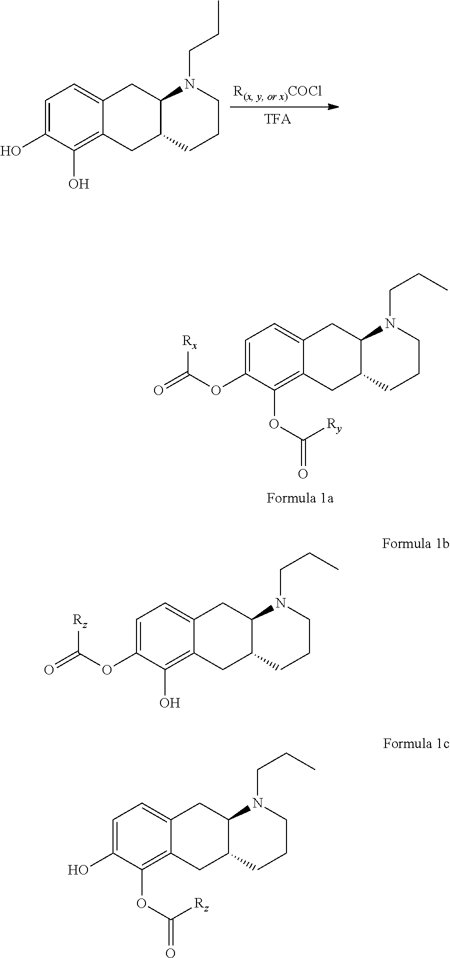

wherein each Rₓ, R_{y}, and R_{z} is independently C₁₋₆ alkanoyl, cycloalkylalkyl, phenylacetyl or benzoyl, or a pharmaceutically acceptable salt thereof, and a pharmaceutically acceptable carrier. Briefly, the catechol amine was treated with acylchloride using TFA as solvent. The crude acyl catecholamine(s) was purified by aluminum oxide chromatography (for a reference on this transformation, see for example: Wikström, Dijkstra, Cremers, Andren, Marchais, Jurva; WO 02/14279). Each of the symmetric, asymmetric and mono-esters described in this example falls within the scope of this invention.

### Example 3

### 2,2-Dimethyl-propionic acid (4aR,10aR)-7-(2,2-dimethyl-propionyloxy)-1-n-propyl-1,2,3,4,4a,5,10,10a-octahydro-benzo[g]quinolin-6-yl ester trifluoroacetate

As a working example, but without limiting the scope of the subject invention, a symmetrical diester was prepared in a similar manner as described above starting from compound 10 (44 mg) and pivaloyl chloride. Yield of Example 3 was 14 mg as a white solid. LC/MS (method 14): RT 2.45 min, ELSD 97.7%, UV 83.9%. MH⁺: 430.2.

### Pharmacological Data

### Example 4

### Pharmacological Testing In Vitro I

D₁ cAMP Assay

The ability of the compounds to either stimulate or inhibit the D₁ receptor mediated cAMP formation in CHO cells stably expressing the human recombinant D₁ receptor was measured as follows. Cells were seeded in 96-well plates at a concentration of 11000 cells/well 3 days prior to the experiment. On the day of the experiment the cells were washed once in preheated G buffer (1 mM MgCl₂, 0.9 mM CaCl₂, 1 mM IBMX (3-i-butyl-1-methylxanthine) in PBS (phosphate buffered saline)) and the assay was initiated by addition of 100 micro-L of a mixture of 30 nM A68930 and test compound diluted in G buffer (antagonism) or test compound diluted in G buffer (agonism).

The cells were incubated for 20 minutes at 37° C. and the reaction was stopped by the addition of 100 micro-L S buffer (0.1 M HCl and 0.1 mM CaCl₂) and the plates were placed at 4° C. for 1 h. 68 micro-L N buffer (0.15 M NaOH and 60 mM NaOAc) was added and the plates were shaken for 10 minutes. 60 micro-1 of the reaction were transferred to cAMP FlashPlates (DuPont NEN) containing 40 micro-L 60 mM Sodium acetate pH 6.2 and 100 micro-L IC mix (50 mM Sodium acetate pH 6.2, 0.1% sodium azide, 12 mM CaCl₂, 1% BSA (bovine serum albumin) and 0.15 micro-Ci/mL ¹²⁵I-cAMP) were added. Following an 18 h incubation at 4° C. the plates were washed once and counted in a Wallac TriLux counter. Compound 10 was demonstrated to act as a D₁ agonist in this assay with an EC₅₀ of 15.5 nM and an intrinsic activity (efficacy) of 100%. In comparison, apomorphine and dopamine were D₁ agonists in this assay with EC₅₀-values of 52 nM and 43 nM, respectively and intrinsic activities (efficacies) of 86% and 100%, respectively.

### Example 5

### Pharmacological Testing In Vitro II

D₂ cAMP Assay

The ability of the compounds to either stimulate or inhibit the D₂ receptor mediated inhibition of cAMP formation in CHO cells transfected with the human D₂ receptor was measured as follows. Cells were seeded in 96 well plates at a concentration of 8000 cells/well 3 days prior to the experiment. On the day of the experiment the cells were washed once in preheated G buffer (1 mM MgCl₂, 0.9 mM CaCl₂, 1 mM IBMX in PBS) and the assay was initiated by addition of 100 micro-1 of a mixture of 1 micro-M quinpirole, 10 microM forskolin and test compound in G buffer (antagonism) or 10 micro-M forskolin and test compound in G buffer (agonism).

The cells were incubated 20 minutes at 37° C. and the reaction was stopped by the addition of 100 microL S buffer (0.1M HCl and 0.1 mM CaCl₂) and the plates were placed at 4° C. for 1 h. 68 microL N buffer (0.15 M NaOH and 60 mM Sodium acetate) were added and the plates were shaken for 10 minutes. 60 micro-L of the reaction were transferred to cAMP FlashPlates (DuPont NEN) containing 40 micro-L 60 mM NaOAc pH 6.2 and 100 micro-L IC mix (50 mM NaOAc pH 6.2, 0.1% Sodium azide, 12 mM CaCl₂, 1% BSA and 0.15 micro-Ci/ml ¹²⁵I-cAMP) were added. Following an 18 h incubation at 4° C. the plates were washed once and counted in a Wallac TriLux counter. Compound 10 was demonstrated to act as a D₅ agonist in this assay with an EC₅₀ of 0.11 nM and an intrinsic activity (efficacy) of 100%. In comparison, apomorphine and dopamine were D₂ agonists in this assay with EC₅₀-values of 3.9 nM and 21 nM, respectively and intrinsic activities (efficacies) of 100% for both compounds.

### Example 6

### Pharmacological Testing In Vitro III

### D₅ Assay

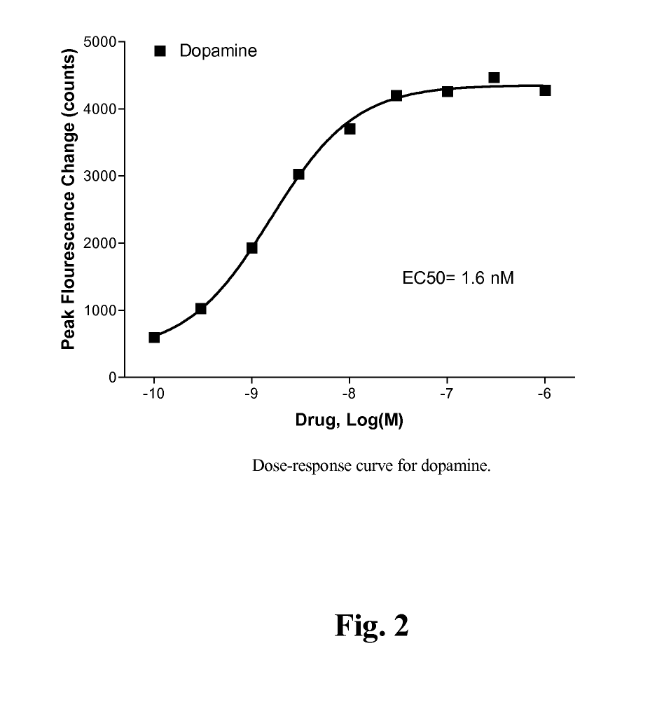

Concentration-dependent stimulation of intracellular Ca²⁺ release by dopamine in hD₅-transfected CHO-Ga16 cells. The cells were loaded with fluoro-4, a calcium indicator dye, for 1 h. Calcium response (fluorescence change) was monitored by FLIPR (fluorometric imaging plate reader) for 2.5 min. Peak responses (EC₅₀) were averaged from duplicate wells for each data point and plotted with drug concentrations (cf. FIG. 2 for dopamine). Compound 10 was demonstrated to act as a D₅ agonist in this assay with an EC₅₀ of 0.06 nM and an intrinsic activity (efficacy) of 95%. In comparison, apomorphine and dopamine were D₅ agonists in this assay with EC₅₀-values of 0.36 nM and 1.6 nM, respectively and intrinsic activities (efficacies) of 88% and 100%, respectively.

### Example 7

### Pharmacological Testing In Vivo I

### D1/D2 Dissections

Dopamine agonists can have activity at either the D1 receptors, the D2 receptors, or both. We have used the rotation response in rats with unilateral 6-OHDA lesions to assess compounds for their ability to stimulate both receptor types and induce rotation [Ungerstedt and Arbuthnott, Brain Res. 1970, 24, 485; Setler et al., Eur. J. Pharmacol. 1978, 50 (4), 419; and Ungerstedt et al. “Advances in Dopamine Research” (Kohsaka, Ed.), Pergamon Press, Oxford, p. 219 (1982)]. 6-OHDA (6-hydroxydopamine) is a neurotoxin used by neurobiologists to selectively kill dopaminergic neurons at the site of injection in the brain in experimental animals. In the 6-OHDA model the nigrostraital dopamine cells are destroyed on one side of the brain (unilateral) by injecting 6-OHDA into the median forebrain bundle, located in front of the substantia nigra. The effects of the unilateral lesion combined with the administration of dopamine agonists such as apomorphine will induce rotation behaviour. Rats weighing 200-250 g were subjected to unilateral 6-OHDA lesions. Animals were permitted minimum three weeks to recover before being tested for rotation response to amphetamine (2.5 mg/kg subcutaneously) and only animals that responded by ipsolateral rotations were used in subsequent dyskinesia studies (examples 8 and 9). Amphetamine increases dopamine levels in the synapse by blocking reuptake and increasing release from presynaptic terminals. This effect is greater in the unlesioned side causing the animals to rotate in the opposite direction as compared to their response to direct agonists such as L-DOPA and apomorphine that act predominantly on the lesioned side of the brain. For D1/D2 in vivo dissection studies were trained on apomorphine (0.1 mg/kg subcutaneously) before being using in experiments and only animals that repeatedly rotated at least 350 times in 90 min were included. Rats where then randomly allocated to the three treatment groups balancing the groups for the animals' rotation response to apomorphine (0.1 mg/kg subcutaneously). For dyskinesia studies animals were not trained on apomorphine; instead they were either primed with L-DOPA (example 9) or used ‘drug-naïve’ (example 8). Experiments consist of determining a minimum effective dose (MED) to induce rotation for the compound in question. Once a MED has been determined, a second experiment is performed to determine the MED of the compound to overcome Nemonapride block (MED_{Nemonapride}). Nemonapride is a D2 antagonist that blocks the D2 receptors, therefore any observed rotations would be dependent upon activity at the D1 receptors. Finally, once the MED_{Nemonapride} is known a third experiment is run using the MED_{Nemonapride} dose and observing the effect of the D1 antagonist, SCH 23390 alone, the D2 antagonist, Nemonapride alone and finally, the effect of combined treatment with SCH 23390 and Nemonapride. This third experiment confirms the activity of the compound at both receptors as either antagonist alone can only partially inhibit the rotation response induced by the test compound while the combination treatment completely blocks all rotations in the rats [Arnt and Hyttel, Psychopharmacology, 1985, 85 (3), 346; and Sonsalla et al., J. Pharmacol Exp. Ther., 1988, 247 (1), 180]. This model was validated using Apomorphine as the proof-of-principle compound for mixed D1/D2 agonists. Compound 10 (administered subcutaneously) had a mixed D1/D2 ratio of about 2 in this model as compared to apomorphine that had a ratio of about 3. A D1 component could not be observed for D2-agonists as exemplified by pramipexole and rotigotine. The data are summarized in Table 1.

TABLE 1
MED and MED_{Nemonapride} for apomorphine, pramipexole, rotigotine, and
compound 10 (all compounds dosed SC).

| | apomorphine | rotigotine | pramipexole | compound 10 |
|---|---|---|---|---|
| MED | 0.010 mg/kg | 0.030 mg/kg | 0.1 mg/kg | 0.00065 mg/kg |
| MED_{Nemonapride} | 0.030 mg/kg | 0.30 mg/kg* | 1.0 mg/kg* | 0.0013 mg/kg |

*Rotations could not be blocked by administration of SCH23390.

Compound 10 has the in vivo profile of a long-lasting dual D1/D2 agonist with a fast onset of action (when dosed buccally or s.c.). Thus, it would be expected that compound 10 could be useful in treating ON/OFF fluctuations in Parkinson's Disease. It may also be used as a ‘rescue drug’ for the OFF periods (freezing).

### Example 8

### Pharmacological Testing In Vivo II

Dyskinesia Model with Naïve 6-OHDA Rats

Twenty rats with unilateral 6-OHDA lesions [see example 7 for experimental details] were used to test induction of dyskinesia by compound 10 (administered subcutaneously; n=7; group 1) compared to L-DOPA/benserazide (6 mg/kg/15 mg/kg subcutaneously; n=7; group 2) and apomorphine (1 mg/kg subcutaneously; n=6; group 3). Benserazide is a DOPA decarboxylase inhibitor which is unable to cross the blood-brain barrier; it is used to prevent metabolism of L-DOPA to dopamine outside the brain.

During the actual dyskinesia experiments, rats received once daily injections of the test compounds subcutaneously and were observed for 3 h following injection. Each animal was observed for 1 minute every 20 min throughout the 3 h period for the presence of dyskinesias using the Abnormal Involuntary Movement Scale (AIMS) as described previously (Lundblad et al., Eur. J Neurosci., 15, 120 (2002)). Rats received drug for 14 consecutive days and were scored on days 1, 2, 3, 4, 5, 8, 10 and 12. Two-way repeated measures ANOVA revealed that there was a significant treatment effect, time effect and treatment by time interaction (p<0.001, in all cases). Post hoc comparisons using Holm-Sidak method indicates that animals treated with compound 10 had significantly less dyskinesia (scores of about 30) compared to animals treated with either L-DOPA or apomorphine (scores of about 65). There were no differences between L-DOPA and apomorphine treated groups. Following this experiment all rats were given subcutaneous injections of compound 10 from day 15-19 in order to determine how compound 10 influenced the severity of dyskinesia seen in the apomorphine and L-DOPA groups. Dyskinesia scoring was performed on day 19 of the experiment (corresponding to 5 days on compound 10). The data showed a partial reversal of the dyskinesias induced by L-DOPA and apomorphine to about the level of dyskinesias induced by compound 10 (which did not cause an increase in dyskinesia in group 1 as compared to the score of about 30 observed after 12 days of treatment). The data are presented in Table 2.

TABLE 2
Induction of dyskinesias by compound 10, L-DOPA, and apomorphine as
well as reduction of dyskinesias induced by L-DOPA or apomorphine by
treatment with compound 10.

| | group 1 | group 2 | group 3 |
|---|---|---|---|
| dose (once daily | compound 10 | L-DOPA/ | apomorphine |
| on days 1-14) | 0.0013 mg/kg | Benserazide | 1 mg/kg SC |
| | SC | 6/15 mg/kg SC | |
| mean AIM score | 27 | 66 | 61 |
| (days 1-12) | | | |
| dose (once daily | compound 10 | compound 10 | compound 10 |
| on days 15-19) | 0.0013 mg/kg | 0.0013 mg/kg SC | 0.0013 mg/kg SC |
| | SC | | |
| mean AIM score | 25 | 18 | 39 |
| (day 19) | | | |

### Example 9

### Pharmacological Testing In Vivo III

### Reversal of L-DOPA-Induced Dyskinesias in 6-OHDA Rats

A separate dyskinesia study addressed the reversal of L-DOPA induced dyskinesias with either pramipexole or Compound 10. Briefly, 18 animals were treated with L-DOPA/Benserazide (6/15 mg/kg subcutaneously) for 7 days. Animals were observed on Days 1, 3 and 5 and AIMS were scored. The day 5 scores were then used to separate the animals into three groups of 6 animals each. Group 1 continued with daily L-DOPA treatment. Group 2 was treated with compound 10 (administered subcutaneously). Group 3 was treated with pramipexole (0.16 mg/kg subcutaneously). Treatment continued daily for 10 days and the amount of dyskinesia was scored on days 1, 5, 9 and 10. Two-way repeated measures analysis of variance indicated that animals treated with compound 10 had significantly fewer dyskinesias than both the pramipexole group and the L-DOPA/Benserazide group. The pramipexole group had significantly less dyskinesias than the L-DOPA/Benserazide group. Hence, compound 10 had a superior profile over pramipexole in terms of reversing dyskinesias induced by L-DOPA. The data are presented in Table 3.

TABLE 3
Reduction of L-DOPA induced dyskinesias by treatment with compound
10 or Pramipexole.

| | group 1 | group 2 | group 3 |
|---|---|---|---|
| dose (once daily | L-DOPA/ | compound 10 | pramipexole |
| on days 1-10) | Benserazide | 0.0013 mg/kg SC | 0.16 mg/kg SC |
| | 6/15 mg/kg SC | | |
| mean AIM score | 75 | 44 | 58 |
| (days 1, 5, 9, 10) | | | |

Accordingly, it is expected that dyskinesias in moderate to severe PD based on L-DOPA-like efficacy and reversal of dyskinesias can be treated by administration of compound 10.

### Example 10

### Pharmacological Testing In Vivo IV

### Superiority Model

Apomorphine and L-DOPA are able to reverse motility deficits in a mouse model of severe dopamine depletion. Both Apomorphine and L-DOPA stimulate D1 and D2 dopamine receptors. Pramipexole, an agonist at D2 receptors is ineffective in this model. Compound 10 has been tested in this model and exhibits a profile similar to Apomorphine and L-DOPA in that they are able to restore locomotion in the mice. In this way, compound 10 is ‘superior’ to other compounds, such as Pramipexole that target D2 receptors only. Bromocriptine is another example of a D2 agonist that does not reverse the deficits in this animal model.

The experiments were performed as follows: Mice previously treated with MPTP (2×15 mg/kg subcutaneously) and that had stable lesions were used and vehicle treated mice served as normal controls. MPTP (1-methyl-4-phenyl-1,2,3,6-tetrahydropyridine) is a neurotoxin that causes permanent symptoms of Parkinson's disease by killing certain neurons in the substantia nigra of the brain. It is used to study the disease in monkeys and mice. On the day of the experiment, mice were treated with AMPT (250 mg/kg subcutaneously) and then returned to their home cages for 1.5 hours after which they were placed in individual cages in the motility unit. AMPT (alpha-methyl-p-tyrosine) is a drug that temporarily reduces brain catecholamine activity (in this case especially dopamine levels). Three hours after the AMPT injection, rescue of locomotive deficits was attempted with compound 10 and activity was recorded for an additional 1.5 hours. The first 30 min of data collected after the rescue treatment was ‘contaminated’ due to stressing the animals with handling and injection as evidenced by increased levels in the vehicle controls therefore the data were analyzed using the last 1 hour of recorded data. Various compounds (all dosed subcutaneously) were tested for their ability to reverse the motility deficits produced in this model. L-DOPA/Benserazide, apomorphine, and compound 10 restored locomotion in the mice in a dose-dependent manner. In contrast, the D2 agonists, pramipexole and bromocriptine did not. The data are presented in Tables 4a-4-e.

TABLE 4a
L-DOPA/Benserazide reverses hypomotility in the MPTP/AMPT mouse
model.

| pretreatment (−1.5 h) | vehicle | AMPT | AMPT |
|---|---|---|---|
| | | 250 mg/kg SC | 250 mg/kg SC |
| treatment (0 h) | vehicle | vehicle | L-DOPA and |
| | | | benserazide |
| activity count (0.5-1.5 h) | 365 | 44 | 50/50 mg/kg SC |
| | | | 676 |

TABLE 4b
Apomorphine reverses hypomotility in the MPTP/AMPT mouse model.

| pretreatment (−1.5 h) | vehicle | AMPT | AMPT |
|---|---|---|---|
| | | 250 mg/kg SC | 250 mg/kg SC |
| treatment (0 h) | vehicle | vehicle | apomophine |
| | | | 1.0 mg/kg SC |
| activity count (0.5-1.5 h) | 694 | 1 | 912 |

TABLE 4c
Compound 10 reverses hypomotility in the MPTP/AMPT mouse model

| | | | |
|---|---|---|---|
| pretreatment | vehicle | AMPT | AMPT |
| (−1.5 h) | | 250 mg/kg SC | 250 mg/kg SC |
| treatment (0 h) | vehicle | vehicle | compound 10 |
| | | | 0.003 mg/kg SC |
| activity count | 405 | 12 | 8 |
| (0.5-1.5 h) | | | |
| pretreatment | AMPT | AMPT | |
| (−1.5 h) | 250 mg/kg SC | 250 mg/kg SC | |
| treatment (0 h) | compound 10 | compound 10 | |
| | 0.01 mg/kg SC | 0.03 mg/kg SC | |
| activity count | 228 | 440 | |
| (0.5-1.5 h) | | | |

TABLE 4d
Bromocriptine does not reverse hypomotility in the MPTP/AMPT
mouse model.

| | | | |
|---|---|---|---|
| pretreatment (−1.5 h) | vehicle | AMPT | AMPT |
| | | 250 mg/kg SC | 250 mg/kg SC |
| treatment (0 h) | vehicle | vehicle | bromocriptine |
| | | | 1 mg/kg SC |
| activity count | 336 | 16 | 25 |
| (0.5-1.5 h) | | | |
| pretreatment (−1.5 h) | AMPT | AMPT | |
| | 250 mg/kg SC | 250 mg/kg SC | |
| treatment (0 h) | bromocriptine | bromocriptine | |
| | 5 mg/kg SC | 10 mg/kg SC | |
| activity count | 17 | 36 | |
| (0.5-1.5 h) | | | |

This model was used to evaluate whether or not Compound 10 exhibits the same superiority as L-DOPA and apomorphine over D2 agonists. A dose response experiment for compound 10 was performed and there was a dose-dependent reversal of the hypomotility deficits induced by severe depletion of endogenous dopamine. A final experiment directly comparing the effects of apomorphine, pramipexole and compound 10 in this model was performed and confirmed that compound 10 was able to restore locomotion in MPTP mice treated and was superior to Pramipexole in this model. The data is presented in Table 4e.

TABLE 4e
Superiority of apomorphine and compound 10 over Pramipexole in the
mouse MPTP/AMPT model.

| | | | |
|---|---|---|---|
| pretreatment | vehicle | AMPT | AMPT |
| (−1.5 h) | | 250 mg/kg SC | 250 mg/kg SC |
| treatment (0 h) | vehicle | vehicle | apomorphine |
| | | | 1 mg/kg SC |
| activity count | 509 | 2 | 904 |
| (0.5-1.5 h) | | | |
| pretreatment | AMPT | AMPT | |
| (−1.5 h) | 250 mg/kg SC | 250 mg/kg SC | |
| treatment (0 h) | pramipexole | compound 10 | |
| | 1 mg/kg SC | 0.030 mg/kg SC | |
| activity count | 176 | 690 | |
| (0.5-1.5 h) | | | |

Based on the above data in Tables 4a-4-e, and in one embodiment of the invention, it is expected that compound 10 can be used to treat a ‘moderate-to-severe PD’ or ‘severe PD’ patient population.

The lower induction of dyskinesias by compound 10 relative to apomorphine and L-DOPA combined with the D1/D2 dissection study (and the MPTP/AMPT mouse+MPTP marmosets studies) supports first-line treatment with compound 10. Today, D2 agonists such as pramipexole are preferred first-line medication due to their better ‘fluctuation side-effects’ profile (e.g. dyskinesias) as compared to L-DOPA. Our data demonstrates that compound 10 is as efficacious as L-DOPA (and apomorphine) but that it also has a better dyskinesia profile than L-DOPA and apomorphine. Since L-DOPA is consistently more efficacious than D2 agonists like pramipexole in all stages of PD, it is believed that compound 10 would be an optimal drug for first-line treatment based on the combined dual D1/D2 profile in vivo, efficacy on par with L-DOPA and better than D2 agonist, and with a dyskinesia profile better than L-DOPA.

### Example 11

### Pharmacological Testing In Vivo V

### Anti-Parkinsonian Effects in MPTP-Treated Common Marmosets

The experiments were conducted using 6 MPTP treated marmosets (2.0 mg/kg daily for up to 5 consecutive days dissolved in sterile 0.9% saline solution). All the animals had previously been treated with L-DOPA (12.5 mg/kg p.o., plus carbidopa 12.5 mg/kg p.o.) administered daily for up to 30 days in order to induce dyskinesia. Prior to the study all subjects exhibited stable motor deficits including a marked reduction of basal locomotor activity, poor coordination of movement, abnormal and/or rigid posture, reduced alertness and head checking movements. Domperidone was administered 60 min before any of the test compounds. Domperidone is an antidopaminergic drug that suppresses nausea and vomiting. Locomotor activity was assessed using test cages that are comprised of 8 photo-electric switches comprised of 8 infra-red beams which are strategically placed in the cage and interruption of a beam is recorded as one count. The total number of beam counts per time segment is then plotted as time course or displayed as area under the curve (AUC) for total activity. The assessment of motor disability was performed by a trained observer blinded to the treatment.

L-DOPA (12.5 mg/kg, p.o.) increased locomotor activity and reversed motor disability as previously described (Smith et al., Mov. Disord. 2002, 17 (5), 887). The dose chosen for this challenge is at the top of the dose response curve for this drug. Compound 10 (administered subcutaneously (0.001 or 0.01 mg/kg SC) produced a dose-related increase in locomotor activity and reversal of motor disability tending to produce in a response greater than for L-DOPA (12.5 mg/kg, p.o.). Compound 10 produced prolonged reversal of motor disability compared to L-DOPA and was as efficacious as L-DOPA. This data is presented in Table 5.

TABLE 5
Mean disability scores of MPTP-marmosets when treated with L-DOPA
or compound 10.

| | group 1 | group 2 | group 3 | group 4 |
|---|---|---|---|---|
| treatment | vehicle | L-DOPA | compound 10 | compound 10 |
| | | 12.5 mg/kg | 0.001 mg/kg | 0.01 mg/kg SC |
| | | PO | SC | |
| disability score | 13.0 | 10.0 | 14.3 | 10.0 |
| (60 min) | | | | |
| disability score | 11.0 | 2.0 | 2.3 | 2.2 |
| (120 min) | | | | |
| disability score | 11.0 | 1.8 | 2.5 | 2.0 |
| (180 min) | | | | |
| disability score | 12.7 | 3.2 | 3.0 | 2.2 |
| (240 min) | | | | |
| disability score | 12.2 | 5.0 | 2.5 | 2.7 |
| (300 min) | | | | |
| disability score | 13.0 | 9.7 | 6.5 | 2.3 |
| (360 min) | | | | |
| disability score | 13.3 | 11.0 | 8.5 | 2.7 |
| (420 min) | | | | |

### Example 12

### Pharmacological Testing In Vivo VI

### Reversal of Reserpine-Induced Hypomotility by Buccal Delivery of Compound 10

Rats weighing ca. 200 g were treated with reserpine (5 mg/kg subcutaneously as a solution in 20% aqueous solutol for which pH was adjusted to 4 with methanesulfonic acid). Administering reserpine to rats depletes presynaptic nerve endings from dopamine and therefore reserpinesed rats are temporarily ‘parkinsonian’ and unable to move unless treated with a dopamine agonist or L-DOPA. A separate group of four animals was treated subcutaneously with the vehicle used for reserpine (group 1). After 23-24 hours the 24 reserpine animals were divided into groups 2-6 with four animals in each. These were treated as summarized below, before they were placed in activity boxes equipped with photosensors and their locomotor activity was recorded over 3 hours. Group 1: treated with 20% ethanol in 0.7% aqueous sodium chloride subcutaneously. Group 2: treated with apomorphine (1 mg/kg administered subcutaneously as an aqueous solution with pH=4. 0.02% ascorbic acid had been added to prevent decomposition of apomorphine). Group 3: treated with compound 10 (administered subcutaneously as solution in 20% ethanol in 0.7% aqueous sodium chloride). Groups 4-6: treated with increasing doses of compound 10 (administrated buccally in the upper right gingival as a solution in 20% ethanol in 0.7% aqueous sodium chloride). The data showed that apomorphine (1 mg/kg subcutaneously; positive control) and compound 10 (administered subcutaneously) reversed the reserpine-induced hypomotility. Compound 10 (administered buccally) reversed the hypomotility. The data is summarized in Table 6.

TABLE 6
Effect of apomorphine (dosed subcutaneously) and compound 10 (dosed
buccally) in the Ungerstedt model.

| | group 1 | group 2 | group 3 |
|---|---|---|---|
| treatment (23-24 h prior | vehicle | reserpine | reserpine |
| to activity measurement) | | 5 mg/kg SC | 5 mg/kg SC |
| treatment (0 h prior to | vehicle | apomorphine | compound 10 |
| activity measurement) | | 1 mg/kg SC | 0.01 mg/kg SC |
| activity count | 486 | 440 | 308 |
| | group 4 | group 5 | group 6 |
| treatment (23-24 h prior | reserpine | reserpine | reserpine |
| to activity measurement) | 5 mg/kg SC | 5 mg/kg SC | 5 mg/kg SC |
| treatment (0 h prior to | vehicle | compound 10 | compound 10 |
| activity measurement) | buccally | 0.05 mg/kg | 0.10 mg/kg |
| | | buccally | buccally |
| activity count | 17 | 378 | 533 |

### Example 13

### Pharmacological Testing In Vivo VII

### Induction of Rotation Response in 6-OHDA Rats by Buccal Delivery of Compound 10

We have used rats with unilateral 6-OHDA lesions to assess compound 10 for its ability to induce rotation after buccal administration [for details on the model, see the description under example 7]. A group of eight animals was treated with apomorphine (positive control; 0.1 mg/kg administered subcutaneously as an aqueous solution with pH=4. 0.02% ascorbic acid had been added to prevent decomposition of apomorphine). Another two groups of eight animals were treated with two different doses of compound 10 (administered buccally in the upper right gingiva as a solution in 20% ethanol in 0.7% aqueous sodium chloride). Apomorphine induced rotations after subcutaneous administration. Buccal delivery of compound 10 also induced circling behavior. The data is summarized in Table 7.

TABLE 7
Effect of apomorphine (dosed subcutaneously) and compound 10 (dosed
buccally) in the Ungerstedt model.

| | apomorphine | compound 10 | compound 10 |
|---|---|---|---|
| dose | 0.1 mg/kg SC | 0.01 mg/kg | 0.1 mg/kg |
| | | buccally | buccally |
| mean number of | 1123 | 910 | 1203 |
| rotations over 3 h | | | |

### Example 14

### Pharmacological Testing In Vivo VIII

### Intravenous and Buccal Pharmacokinetic Study in the Minipig

The objective of this study was to determine the plasma concentrations of compound 10 in minipig following dosing with compound 10 (by either intravenous administration at 0.0025 mg/kg or by buccal administration at 0.010 mg/kg and 0.040 mg/kg).

### Study Design

### Test and Control Articles

The test article was compound 10. The vehicles for the test article were Sterile saline (0.9% NaCl) (intravenous administration) supplied by Baxter, Norfolk or Ascorbic acid reconstituted in Water for Injection (buccal administration) supplied by VWR International, Leicestershire. Formulations were prepared on the day of dosing.

### Test System and Dose Levels

Three male minipigs of the Göttingen ApS strain were supplied by Ellegaard Göttingen, Dalmose, Denmark. At initiation of dosing, animals were approximately 15 to 17 weeks old. Each animal was dosed once on three separate occasions according to the following study design:

| | Group | Dose | | Occasion | | Animal |
|---|---|---|---|---|---|---|
| Group | des- | level | Dose | (Study | | numbers |
| number | cription | (μg/kg) | volume | Day) | Route | Male |
| 1 | Low | 2.5 | 0.5 mL/kg | 1 | Intra- | 1-3 |
| | | | | | venous | |
| | | | | | (Bolus) | |
| 1 | Low | 10 | 10 μL/kg | 3 | Buccal | 1-3 |
| 1 | High | 40 | 10 μL/kg | 5 | Buccal | 1-3 |

Animals were deprived of food overnight and anaesthetised with isoflurane in oxygen (administered by facemask), prior to each dosing occasion.

### Day 1—Intravenous Administration

Intravenous administrations were performed by slow manual injection via a temporary catheter placed in the ear vein whilst under anaesthesia, animals were allowed to recover from the anaesthesia immediately after dosing. Whilst anaesthetised, a catheter was inserted into the jugular vein and secured in place for the purpose of blood collection. The catheter was filled with heparin (250 iu/mL in 0.9% sodium chloride). The exterior portion of the catheter was routed from the ventral neck to the dorsum of the minipig and protected by bandaging. The distal end of the catheter was capped and placed in a re-sealable pouch within the bandage. The jugular catheter was retained in place and flushed with heparinised saline every 24 hours.

### Days 3 and 5—Buccal Administrations

Buccal administrations were performed by applying the test formulation to the buccal membrane for 5 minutes while the animal was anaesthetised. Any residual formulation remaining in the mouth after the 5 minute application was left in the mouth. Animals were allowed to recover from the anaesthesia immediately after dosing.

### Plasma Concentrations

Blood samples were taken from all animals on Day 1 following intravenous (bolus) administration, all animals on Day 3 following buccal administration of a low dose and all animals on Day 5 following buccal administration of a high dose for pharmacokinetic analysis. The samples (1.0 mL) were collected from the jugular vein (via catheter) into tubes containing EDTA anticoagulant. Prior to addition of the blood sample, 100 microL of a stabiliser (2% beta-mercaptoethanol containing 20 mg/mL ascorbic acid) was added to each pot. The stabiliser was prepared fresh on each day of sample collection. Samples were collected as follows:
Day 1: 5, 10, 15, 30 and 45 minutes and 1, 2, 4, 6, 8, 12 and 16 hours post-dose
Day 3: pre-dose and at 5, 10, 15, 30 and 45 minutes and 1, 2, 4, 6, 8, 12, 16 and 24 hours post-dose
Day 5: pre-dose and at 5, 10, 15, 30 and 45 minutes and 1, 2, 4, 6, 8, 12, 16 and 24 hours post-dose

The times of the blood sampling were generally adhered to. The greatest deviation from the scheduled timepoints was one minute late at the 5 minute timepoint on Day 3. The blood samples were centrifuged within one hour of sample collection and the resultant plasma was frozen prior to analysis.

### Sample Preparation Procedure

| Step | Procedure |
|---|---|
| 1 | Thaw frozen quality control samples, control matrix and matrix study |
| | samples and calibration standards at room temperature. |
| 2 | Vortex mix samples (ca. 10 seconds). |
| 3 | Centrifuge (ca. 10 minutes, ca. 3500 rpm, room temperature) in the |
| | bench top centrifuge or corresponding ‘g’ force in a micro-centrifuge. |
| 4 | Aliquot calibration standards, QCs, study samples and blanks (100 |
| | μL)* into a 2 mL 96 deep well plate. |
| 5 | Return unused portion of samples to freezer. |
| 6 | Add internal standard solution (500 μL, solution IS C) using a |
| | repeating pipette, to all wells except blanks, which receive 500 μL of |
| | 100 mM ammonium formate (aq) + 1% formic acid. |
| 7 | Cap the plate and gently mix on a plate mixer (ca. 5 minutes). |
| 8 | Centrifuge the plate in a bench top centrifuge (ca. 3500 rpm, 10 |
| | minutes, room temperature). |
| 9 | Prime SPE plate (Oasis HLB 10 mg) with methanol (500 μL per |
| | well). Use minimum pressure or gravity and do not allow to dry. |
| 10 | Prime plate with water (500 μL per well) using minimum pressure. |
| 11 | Transfer samples (approximately 500 μL) to plate using an automatic |
| | 8 channel pipette. |
| 12 | Pass through plate using minimum pressure. |
| 13 | Wash plate with water:methanol (90:10 v/v) (500 μL) using |
| | minimum pressure and then increase pressure to maximum for one |
| | minute. |
| 14 | Slowly elute sample into 1.2 mL 96 deep well plate with 20 mM |
| | ammonium formate (aq):acetonitrile:formic acid (50:50:2 v/v/v) (250 |
| | μL) using minimum pressure and then increase pressure to dry |
| | packing material completely. |
| 15 | Pulse spin the plate containing the eluate to 1000 rpm in a bench-top |
| | centrifuge (place into centrifuge, spin up to 1000 rpm and then stop). |
| 16 | Evaporate the acetonitrile composition of the eluate under a stream of |
| | nitrogen (nominal 30° C.) for a minimum of 30 minutes and until an |
| | estimated half of the original volume remains |
| 17 | Add 100 μL of (20 mM ammonium formate (aq) + 0.5% formic |
| | acid):acetonitrile (90:10 v/v) containing 4 mg/mL ascorbic acid to |
| | each well. |
| 18 | Cap the plate and vortex mix (ca. 2 minutes). |
| 19 | Centrifuge the plate in a bench top centrifuge (ca. 3500 rpm, 10 |
| | minutes, nominal room temperature). |
| 20 | Submit for analysis. |

### Analytical Methods

The plasma concentrations of compound 10 were determined after solid phase extraction of the plasma samples followed by high performance liquid chromatography with tandem mass spectrometric detection (LC-MS/MS) using a sample volume of about 100 microL.

Internal standard solution, containing the internal standard of compound 10 was added to thawed plasma samples (100 microL aliquot). The SPE plate (Oasis HLB, 10 mg) was conditioned with methanol (500 microL) followed by water (500 microL). The sample (approx. 500 microL aliquot) was transferred to the pre-conditioned SPE plate. The sample was then passed through the cartridge, which was then washed with water:methanol (90:10 v/v, 0.5 mL). The sample was then eluted into a fresh 96 well polypropylene collection plate with 20 mM ammonium formate (aq): acetonitrile: formic acid (50:50:2 v/v/v, 250 microL). The organic component of the eluted samples was then evaporated under a gentle stream of nitrogen until approximately 50% of the original volume was remaining. An aliquot (100 microL) of a solution containing 20 mM ammonium formate (aq) and 0.5% formic acid:acetonitrile (90:10 v/v) together with 4 mg/mL ascorbic acid was added to the remaining aqueous component of the sample in each well, vortex mixed, centrifuged (3500 rpm, 10 minutes, room temperature) prior to being submitted for UHPLC-MS/MS analysis.

Concentrations of compound 10 in calibration standards, QC samples and study samples were determined using least squares linear regression with 1/x weighting for compound 10. The plasma concentrations of compound 10 were determined after solid phase extraction of the plasma samples followed by high performance liquid chromatography with tandem mass spectrometric detection (LC-MS/MS). The method was validated and has a lower limit of quantification (LLOQ) of 10 pg/mL using 100 microL of plasma.

Analytical Procedure: Liquid chromatography—tandem mass spectrometry (LC-MS/MS) API 5000: Final extract solutions were submitted for LC-MS/MS analysis under the following conditions.

### LC Conditions:

| | |
|---|---|
| Analytical column # | Waters BEH UPLC Phenyl 100 × 2.1 mm |
| | column, 1.7 microm particle size, part |
| | number 186002885 |
| In line filter (Acquity) | Supplier: Waters Part n/o 700002775 |
| Column oven | Nominal 50° C. |
| temperature# | |
| Autosampler | Nominal 4° C. |
| temperature | |
| Mobile phase A# | 20 mM ammonium formate_{(aq)} + 0.5% formic |
| | acid |
| Mobile phase B# | Acetonitrile |
| Flow rate# | 0.5 mL/min |
| Gradient settings: | See table below |

| Time (minutes) | A (%) | B (%) |
|---|---|---|
| 0.0 | 95 | 5 |
| 0.5 | 95 | 5 |
| 6.0 | 65 | 35 |
| 6.1 | 2 | 98 |
| 7.0 | 2 | 98 |
| 7.1 | 95 | 5 |
| 8.0 | 95 | 5 |

| | |
|---|---|
| Switching valve times | 0-1.2 mins - To waste |
| | 1.2-6 mins - To MS |
| | 6-8 mins - To waste |
| Slave pump solvent | (20 mM ammonium formate_{(aq)} + 0.5% |
| | formic acid):acetonitrile (50:50 v/v) |
| Slave pump flow rate | 0.5 mL/min |
| Wash solvent 1# | (20 mM ammonium formate_{(aq)} + 0.5% |
| Weak wash (Acquity) | formic acid):acetonitrile (90:10 v/v) |
| Wash solvent 2# | Water:methanol:TFA (50:50:0.1 v/v/v) |
| Strong wash (Acquity) | |
| Injection mode | partial loop with needle over-fill |
| (Acquity) | |

### LC Conditions

| | |
|---|---|
| Injection loop volume (Acquity) | 50 microL |
| Needle placement | 2.0 mm from bottom |
| Injection volume (Recommended) | 50 microL |

### Waters Acquity

| | |
|---|---|
| Weak wash volume (μL) | 3000 (Range 200 to 5000) |
| Strong wash volume (μL) | 3000 (Range 0 to 5000) |

### Mass Spectrometer Parameters API 5000

| | |
|---|---|
| Mode of operation# | Turbo IonSpray (Positive ion) (MS/MS) |
| Collision gas setting (CAD) | 6 [Where a setting of 12 is approximately |
| | equal to 4.8 × 10−5 Torr for a API 4000 |
| | instrument] |
| Curtain gas setting (CUR) | 20 psi |
| Ion source gas 1 (GS1) | 50 psi |
| Ion source gas 2 (GS2) | 70 psi |
| IonSpray voltage (IS) | 5500 V |
| Temperature (TEM) | 650° C. |
| Q1 Resolution | Unit |
| Q3 Resolution | Low |
| Interface heater status | On |
| Analysis time | 6 minutes in two periods: |
| Period one: | 3.5 minutes |
| Period two: | 2.5 minutes |

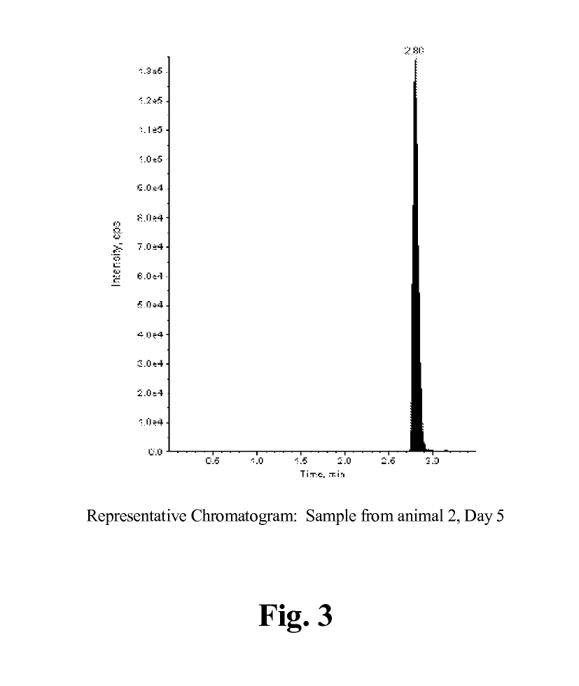

A representative chromatogram generated using the above procedure and acquired during the determination of compound 10 in minipig plasma is presented in FIG. 3. As the quantification of compound 10 was based upon peak height ratios, the integrations on some of the chromatograms include additional noise and interference peaks to ensure the correct peak height is measured.

### Plasma Concentrations of Compound 10 Following Intravenous Administration

Plasma concentrations for compound 10 following single intravenous bolus administration of compound 10 at 0.0025 mg/kg. The data are summarized in Table 8.

TABLE 8
Plasma concentrations of compound 10 in minipigs following intravenous
administration of compound 10 (0.0025 mg/kg)

| | Day 1 | Day 1 | Day 1 | Day 1 | Day 1 | |
|---|---|---|---|---|---|---|
| | Hour | Hour | Hour | Hour | Hour | Day 1 |
| Animal | 0.08 | 0.17 | 0.25 | 0.5 | 0.75 | Hour 1 |
| 1 | 1920 | 1260 | 828 | 536 | 667 | 451 |
| 2 | 1210 | 834 | 740 | 562 | 534 | 379 |
| 3 | 1720 | 976 | 1170 | 922 | 656 | 432 |
| Mean | 1620 | 1020 | 913 | 673 | 619 | 421 |
| (pg/ml) | | | | | | |
| SD (n − 1) | 366 | 217 | 227 | 216 | 73.8 | 37.3 |
| | Day 1 | Day 1 | Day 1 | Day 1 | Day 1 | Day 1 |
| Animal | Hour 2 | Hour 4 | Hour 6 | Hour 8 | Hour 12 | Hour 16 |
| 1 | 198 | 74.8 | 47.6 | 24.5 | 17.0 | 8.58 |
| 2 | 196 | 76.3 | 56.2 | 33.9 | 29.9 | 9.25 |
| 3 | 352 | 115 | 75.6 | 40.7 | 20.9 | 10.1 |
| Mean | 249 | 88.7 | 59.8 | 33.0 | 22.6 | 9.31 |
| (pg/ml) | | | | | | |
| SD (n − 1) | 89.5 | 22.8 | 14.3 | 8.13 | 6.62 | 0.762 |

Following single intravenous bolus administration of compound 10 at 0.0025 mg/kg to male minipigs, maximum plasma concentrations of compound 10 were observed at 5 minutes post-dose, i.e. at the first blood sampling time post intravenous administration. Plasma concentrations of compound 10 appeared to decline in a generally bi-phasic manner with an apparent terminal elimination half-life (t½) ranging from 3.4 to 4.3 hours, with the start of the apparent terminal phase occurring at 4 hours post-dose.

Over the 16 hour sampling period, plasma concentrations were quantifiable (i.e. above the LLOQ of 10 pg/mL) up to 12 hours post-dose in 2 animals, with concentrations estimated at 16 hour post-dose as levels were above 20% of the LLOQ. In one animal (animal 3), plasma concentrations were greater than the LLOQ throughout the 16 hour period.

### Plasma Concentrations of Compound 10 Following Buccal Administration

Plasma concentrations of compound 10 in minipigs following buccal administration (0.010 mg/kg). The data are summarized in Table 9.

TABLE 9
Plasma concentrations of compound 10 in minipigs following buccal administration of
compound 10 (0.010 mg/kg)

| | Day 3 | Day 3 | Day 3 | Day 3 | Day 3 | Day 3 | Day 3 |
|---|---|---|---|---|---|---|---|
| Animal | Hour 0.08 | Hour 0.17 | Hour 0.25 | Hour 0.5 | Hour 0.75 | Hour 1 | Hour 2 |
| 1 | 379 | 105 | 185 | 758 | 1100 | 791 | 501 |
| 2 | 55.2 | 221 | 622 | 556 | 964 | 817 | 372 |
| 3 | 16.1 | 117 | 134 | 943 | 1070 | 1000 | 589 |
| Mean | 150 | 148 | 314 | 752 | 1040 | 869 | 487 |
| (pg/ml) | | | | | | | |
| SD (n − 1) | 199 | 63.8 | 268 | 194 | 71.5 | 114 | 109 |

| | Day 1 | Day 1 | Day 1 | Day 1 | Day 1 | Day 1 |
|---|---|---|---|---|---|---|
| Animal | Hour 4 | Hour 6 | Hour 8 | Hour 12 | Hour 16 | Hour 24 |
| 1 | 98.3 | 84.5 | 46.7 | 26.8 | 6.91 | <2.00 |
| 2 | 91.7 | 26.5 | 26.1 | 23.4 | NR | 4.94 |
| 3 | 206 | 75.8 | 55.4 | 157 | NS | 26.1 |
| Mean | 132 | 62.3 | 42.7 | 69.1 | 6.91 | 15.5 |
| (pg/ml) | | | | | | |
| SD (n − 1) | 64.2 | 31.3 | 15.0 | 76.2 | | |

NR: No result reported
NS: No Sample

Plasma concentrations of compound 10 in minipigs following buccal administration (0.040 mg/kg). The data are summarized in Table 10.

TABLE 10
Plasma concentrations of compound 10 in minipigs following buccal
administration of compound 10 (0.040 mg/kg).

| | Day 5 | Day 5 | Day 5 | Day 5 | Day 5 | Day 5 | Day 5 |
|---|---|---|---|---|---|---|---|
| Animal | Hour 0.08 | Hour 0.17 | Hour 0.25 | Hour 0.5 | Hour 0.75 | Hour 1 | Hour 2 |
| 1 | 695 | 1930 | 3330 | 8230 | 10700 | 10800 | 6340 |
| 2 | 427 | 1820 | 3890 | 5310 | 10500 | 9580 | 3880 |
| 3 | 2750 | 6060 | 9140 | 7880 | 15900 | 11900 | 8680 |
| Mean | 1290 | 3270 | 5450 | 7140 | 12400 | 10760 | 6300 |
| (pg/ml) | | | | | | | |
| SD (n − 1) | 1270 | 2420 | 3210 | 1590 | 3060 | 1160 | 2400 |

| | Day 1 | Day 1 | Day 1 | Day 1 | Day 1 | Day 1 |
|---|---|---|---|---|---|---|
| Animal | Hour 4 | Hour 6 | Hour 8 | Hour 12 | Hour 16 | Hour 24 |
| 1 | 2560 | 781 | 445 | 272 | 142 | 44.3 |
| 2 | 746 | 278 | 212 | 139 | 58.4 | 78.9 |
| 3 | 2490 | 1730 | 910 | 1400 | 677 | 233 |
| Mean | 1930 | 930 | 522 | 604 | 292 | 119 |
| (pg/ml) | | | | | | |
| SD (n − 1) | 1030 | 737 | 355 | 693 | 336 | 100 |

Following single buccal administration of compound 10 at 0.010 mg/kg and 0.040 mg/kg to the male minipig, compound 10 was rapidly absorbed, with compound 10 being quantifiable in plasma at 5 minute post-dose. Maximum plasma concentrations were observed at about 0.75 hours post-dose, with the exception of animal 1 at the 0.040 mg/kg dose level with a delayed tmax of 1 hour post-dose. After attainment of Cmax, plasma concentrations of compound 10 appeared to decline in a bi-phasic manner, with mean apparent terminal half-lives of 5.1 and 5.6 hours at the 0.010 mg/kg and 0.040 mg/kg dose levels, respectively.

Over the 24 hour sampling period, plasma levels of compound 10 remained above the LLOQ, apart from 2 animals following the 0.010 mg/kg dose where plasma concentrations were either estimated (as levels were above 20% of the LLOQ; 16 hour post-dose for 1M; 24 hour post-dose for 2M), or were not quantifiable (being <20% of LLOQ; 24 h post-dose for 1M).

### Dose Proportionality

Fold increases in systemic exposure to compound 10 following increases in dose from 0.010 mg/kg and 0.040 mg/kg compound 10 are presented below.

| | 10 μg/kg | 40 μg/kg |
|---|---|---|
| Dose | Males | Males |
| Dose Increment | NA | 4.0 |
| Increase in AUC_{0-∞} | NA | 12.1 |
| Increase in AUC_{0-∞} | NA | 18.9 |
| Increase in Cₘₐₓ | NA | 11.9 |

NA = Not applicable

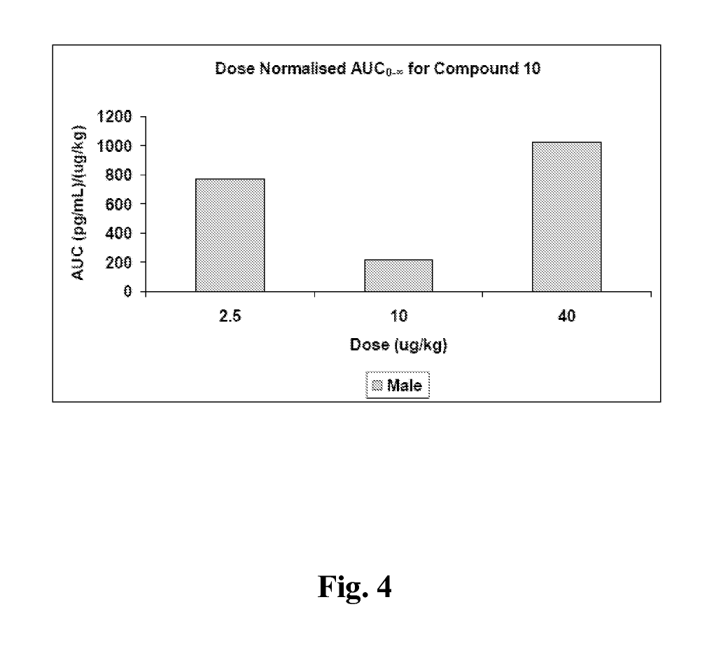

Systemic exposure to compound 10 increased in a supra-proportional manner over the 0.010 mg/kg and 0.040 mg/kg dose range with AUC_{0-∞} and Cₘₐₓ increasing by 12-fold over the 4-fold increase in dose. The bioavailability of compound 10 following buccal administration was dose dependent, ranging from 30 to 42% at 0.010 mg/kg, increasing to 73 to 136% at 0.040 mg/kg The dose normalised AUC0-∞ and Cmax for compound 10 are presented graphically in FIGS. 3 and 4, respectively.

### CONCLUSION

Following intravenous bolus administration of 0.025 mg/kg compound 10 to male minipigs, plasma concentrations of compound 10 appeared to decline in a bi-phasic manner with individual apparent terminal elimination half-life ranging from 3.4 to 4.3 hours.

Absorption of compound 10 was rapid following single buccal administration of compound 10, with maximum plasma concentrations being observed at 0.75 to 1 hours post-dose. Plasma concentrations of compound 10 appeared to decline in a bi-phasic manner and the apparent terminal elimination half-life was independent of dose, with values ranging from 3.1 to 5.6 hours in individual animals.

Following buccal administration, systemic exposure to compound 10 appeared to increase in a supra-proportional manner with a 12-fold increase in both AUC0-∞ and Cmax over the 0.010 to 0.040 mg/kg dose range. Due to the non-linearity in exposure, bioavailability of compound 10 was dose dependent with mean values of 31 to 35% at 0.010 mg/kg increasing to 105 to 122% at 0.040 mg/kg.

### Example 15

### Pharmacological Testing In Vivo IX

### Induction of Circling Behaviour in a Rat Model of Parkinson's Disease by Intranasal Administration of Compound 10

Animals were generated as described under example 7. Four groups of animals were dosed with various doses of compound 10 (group 1, 1 microg/kg; group 2, 10 microg/kg; group 3, 25 microg/kg; group 4, 50 microg/kg). In all cases, compound 10 was administered in one of the nostrils in a volume of 20 microL of a solution of the appropriate concentration in 20% ethanol in 0.7% aqueous sodium chloride containing 0.02% ascorbic acid. The drug solution was applied to one of the nostrils and the nose was gently massaged to ensure distribution of the administered solution over the nasal mucosa. The degree of rotation behaviour of the animals was recorded over the next 3 hours. The data are presented in Table 11.

TABLE 11
Rotation response of unilaterally lesioned 6-OHDA rats over 3 hours
following intranasal administration of compound 10.

| | group 1 | group 2 | group 3 | group 4 |
|---|---|---|---|---|
| mean number of | 401 | 691 | 1286 | 2122 |
| rotations (0-3 h) | | | | |

### Example 16

### Pharmacological Testing In Vivo X

### Induction of Rotation Response in 6-OHDA Rats by Transdermal Delivery of Compound 10

We have used rats with unilateral 6-OHDA lesions to assess compound 10 for its ability to induce rotation after transdermal administration [for details on the model, see the description under example 7]. Three groups of six animals were treated with different doses of compound 10 administered transdermally. Compound 10 (24 mg) was suspended in a mixture of 0.02% ascorbic acid and 20% ethanol in saline (9 mL); the resulting suspension was diluted with dimethyl sulfoxide (0.45 mL). The appropriate amount of this formulation was applied to the ears of the animals. The ears were rubbed gently before the rotation response of the animals was assessed over 3 h. Transdermal delivery of compound 10 induced circling behavior in all three groups. The data is summarized are Table 12.

TABLE 12
Effect of compound 10 (dosed transdermally) in the Ungerstedt model.

| | group 1 | group 2 | group 3 |
|---|---|---|---|
| dose | 0.127 mg/kg | 0.254 mg/kg | 0.381 mg/kg |
| | compound 10 | compound 10 | compound 10 |
| | transdermally | transdermally | transdermally |
| mean number of | 482 | 837 | 1448 |
| rotations over 3 h | | | |

1. A pharmaceutical composition for delivery across the oral mucosa, nasal mucosa or skin comprising (4aR,10aR)-1-n-propyl-1,2,3,4,4a,5,10,10a-octahydro-benzo[g]quinoline-6,7-diol or a pharmaceutically acceptable salt thereof, and a pharmaceutically acceptable carrier.

2. (canceled)

3. The pharmaceutical composition of claim 1 for delivery across the oral mucosa comprising (4aR,10aR)-1-n-propyl-1,2,3,4,4a,5,10,10a-octahydro-benzo[g]quinoline-6,7-diol or a pharmaceutically acceptable salt thereof, and a pharmaceutically acceptable carrier.

4. (canceled)

5. The pharmaceutical composition of claim 3 wherein the delivery across the oral mucosa occurs through oral buccal route, sublingual route or through the lips.

6. (canceled)

7. (canceled)

8. A pharmaceutical composition for intranasal administration comprising (4aR,10aR)-1-n-propyl-1,2,3,4,4a,5,10,10a-octahydro-benzo[g]quinoline-6,7-diol or a pharmaceutically acceptable salt thereof, and a pharmaceutically acceptable carrier.

9. (canceled)

10. The pharmaceutical composition of claim 8 further comprising a permeation enhancer.

11. (canceled)

12. A pharmaceutical composition for transdermal delivery comprising (4aR,10aR)-1-n-propyl-1,2,3,4,4a,5,10,10a-octahydro-benzo[g]quinoline-6,7-diol or a pharmaceutically acceptable salt thereof, and a pharmaceutically acceptable carrier.

13. (canceled)

14. (canceled)

15. (canceled)

16. (canceled)
